SEQUENCE LISTING (16 sequences)

SEQ ID NO: 1 - protein, 250 aa, Artificial Sequence [sequence omitted]

SEQ ID NO: 2 - DNA, 818 bp, Artificial Sequence
  Synthetic Construct
agcagccgtccggagccagccaacgagcggaaaatggcagacaatttttcgctccatgatgcgttatctgggtctggaaacccaaaccctcaaggatggcctggcgcatgggggaaccagcctgctggggcagggggctacccaggggcttcctatcctggggcctaccccgggcaggcacccccaggggcttatcctggacaggcacctccaggcgcctaccatggagcacctggagcttatcccggagcacctgcacctggagtctacccagggccacccagcggccctggggcctacccatcttctggacagccaagtgcccccggagcctaccctgccactggcccctatggcgcccctgctgggccactgattgtgccttataacctgcctttgcctgggggagtggtgcctcgcatgctcataacaattctgggcacggtgaagcccaatgcaaacagaattgctttagatttccaaagagggaatgatgttgccttccactttaacccacgcttcaatgagaacaacaggagagtcattgtttgcaatacaaagctggataataactggggaagggaagaaagacagtcggttttcccatttgaaagtgggaaaccattcaaaatacaagtactggttgaacctgaccacttcaaggttgcagtgaatgatgctcacttgttgcagtacaatcatcgggttaaaaaactcaatgaaatcagcaaactgggaatttctggtgacatagacctcaccagtgcttcatataccatgatataatctgaaaggggcagattaaaaaaaaaaacgga

SEQ ID NO: 3 - protein, 143 aa, Artificial Sequence
  Synthetic Construct
GAPAGPLIVPYNLPLPGGVVPRMLITILGTVKPNANRIALDFQRGNDVAFHFNPRFNENNRRVIVCNTKLDNNWGREERQSVFPFESGKPFKIQVLVEPDHFKVAVNDAHLLQYNHRVKKLNEISKLGISGDIDITSASYTMI

SEQ ID NO: 4 - DNA, 432 bp, Artificial Sequence
  Synthetic Construct
atgggcgcccctgctgggccactgattgtgccttataacctgcctttgcctgggggagtggtgcctcgcatgctcataacaattctgggcacggtgaagcccaatgcaaacagaattgctttagatttccaaagagggaatgatgttgccttccactttaacccacgcttcaatgagaacaacaggagagtcattgtttgcaatacaaagctggataataactggggaagggaagaaagacagtcggttttcccatttgaaagtgggaaaccattcaaaatacaagtactggttgaacctgaccacttcaaggttgcagtgaatgatgctcacttgttgcagtacaatcatcgggttaaaaaactcaatgaaatcagcaaactgggaatttctggtgacatagacctcaccagtgcttcatataccatgata

SEQ ID NO: 5 - DNA, 753 bp, Artificial Sequence
  Synthetic Construct
atggcagataacttctcgctgcatgacgcactgtcgggctcgggtaatccgaatccgcagggctggccgggcgcttggggtaatcaaccggcaggtgccggcggttatccgggtgcttcttatccgggcgcatacccgggtcaggctccgccgggtgcatacccgggtcaagcaccgccgggtgcatatcatggtgcaccgggtgcttacccgggtgcaccggctccgggtgtgtatccgggtccgccgtcaggcccgggtgcctacccgagctctggtcagccgtcggcaccgggtgcatatccggcaacgggtccgtacggtgcaccggcaggtccgctgattgttccgtataacctgccgctgccgggcggtgtggttccgcgtatgctgattaccatcctgggcacggtcaaaccgaacgctaatcgtattgcgctggattttcaacgcggtaacgacgtggcgtttcatttcaacccgcgcttcaatgaaaacaatcgtcgcgtcatcgtgtgcaataccaaactggataacaattggggccgtgaagaacgccagagtgtttttccgttcgaatccggtaaaccgtttaaaatccaagttctggtcgaaccggatcacttcaaagtggccgttaatgacgcacatctgctgcagtataaccaccgtgtcaaaaaactgaatgaaattagtaaactgggcatttctggcgacattgacctgacctcggcgtcctacacgatgatttaa

SEQ ID NO: 6 - DNA, 432 bp, Artificial Sequence
  Synthetic Construct
atgggtgcaccggcaggtccgctgattgttccgtataacctgccgctgccgggcggtgtggttccgcgtatgctgattaccatcctgggcacggtcaaaccgaacgctaatcgtattgcgctggattttcaacgcggtaacgacgtggcgtttcatttcaacccgcgcttcaatgaaaacaatcgtcgcgtcatcgtgtgcaataccaaactggataacaattggggccgtgaagaacgccagagtgtttttccgttcgaatccggtaaaccgtttaaaatccaagttctggtcgaaccggatcacttcaaagtggccgttaatgacgcacatctgctgcagtataaccaccgtgtcaaaaaactgaatgaaattagtaaactgggcatttctggcgacattgacctgacctcggcgtcctacacgatgatt

SEQ ID NO: 7 - protein, 143 aa, Artificial Sequence
  Synthetic Construct
GAPAGPLIVPYNLPLPGGVVPRMLITILGTVKPNANRIALDFQRGNDVAFHFNPRFNENNRRVIVCNTKLDNNWGREERQSVFPFESGKPFKIQVLVEPEHFKVAVNDAHLLQYNHRVKKLNEISKLGISGDIDLTSASYTMI

SEQ ID NO: 8 - protein, 143 aa, Artificial Sequence
  Synthetic Construct
GAPAGPLIVPYNLPLPGGVVPRMLITILGTVKPNANRIALDFQRGNDVAFHFNPRFNENNRRVIVCNTKLDNNWGREERQSVFPFESGKPFKIQVLVEPEHFKVAVNDAHLLQYNHRAKKLNEISKLGISGDIDLTSASYTMI

SEQ ID NO: 9 - protein, 11 aa, Artificial Sequence
  Synthetic Construct
DICLPRWGCLW

SEQ ID NO: 10 - protein, 355 aa, Artificial Sequence
  Synthetic Construct
MLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKAGGSGGSGVPAGPLTVPYDLPLPGGVMPRMLITIMGTVKPNANRIVLDFRRGNDVAFHFNPRFNENNRRVIVCNTKQDNNWGKEERQSAFPFESGKPFKIQVLVEADHFKVAVNDAHLLQYNHRMKNLREISQLGISGDITLTSANHAMI

SEQ ID NO: 11 - DNA, 1080 bp, Artificial Sequence
  Synthetic Construct
catatgttagaaaaatgttgtgctgcagctgatccccacgaatgttacgcaaaggtgtttgacgagtttaagccgctggttgaagaaccgcagaacctgatcaaacaaaactgcgagctgttcgaacagctgggcgagtacaagtttcaaaacgcgctgctggtgcgttataccaagaaagtgccgcaggttagcaccccgaccctggtggaagttagccgtaacctgggtaaagttggcagcaaatgctgcaaacatccggaggcgaagcgtatgccgtgcgcggaagactacctgagcgtggttctgaaccaactgtgcgtgctgcacgagaaaaccccggtgagcgatcgtgttaccaagtgctgcaccgaaagcctggttaaccgtcgtccgtgcttcagcgcgctggaagtggacgaaacctatgttccgaaagagtttaacgcggaaaccttcacctttcacgcggatatctgcaccctgagcgagaaggaacgtcagattaagaaacaaaccgcgctggttgagctggttaagcacaaaccgaaggcgaccaaagaacagctgaaggcggtgatggacgatttcgcggcgtttgttgagaaatgctgcaaggcgggtggcagcggtggcagcggtgtgccggcgggtccgctgaccgttccgtacgacctgccgctgccgggtggcgtgatgccgcgtatgctgatcaccattatgggtaccgtgaaaccgaacgcgaaccgtatcgttctggacttccgtcgtggcaacgatgttgcgttccactttaacccgcgttttaacgagaacaaccgtcgtgtgattgtttgcaacaccaaacaggataacaactggggtaaagaggagcgtcaaagcgcgttcccgtttgagagcggcaaaccgttcaagattcaggtgctggttgaagcggaccactttaaagtggcggttaacgatgcgcacctgctgcaatataaccaccgtatgaagaacctgcgtgaaattagccaactgggcattagcggtgacattaccctgaccagcgcgaaccacgcgatgatttaatgaaagctt

SEQ ID NO: 12 - protein, 355 aa, Artificial Sequence
  Synthetic Construct
MGVPAGPLTVPYDLPLPGGVMPRMLITIMGTVKPNANRIVLDFRRGNDVAFHFNPRFNENNRRVIVCNTKQDNNWGKEERQSAFPFESGKPFKIQVLVEADHFKVAVNDAHLLQYNHRMKNLREISQLGISGDITLTSANHAMIGGSGGSLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKA

SEQ ID NO: 13 - DNA, 1080 bp, Artificial Sequence
  Synthetic Construct
catatgggtgtgccggcgggtccgctgaccgttccgtacgacctgccgctgccgggtggcgtgatgccgcgtatgctgatcaccattatgggtaccgtgaaaccgaacgcgaaccgtatcgttctggacttccgtcgtggcaacgatgttgcgttccactttaacccgcgttttaacgagaacaaccgtcgtgtgattgtttgcaacaccaaacaggataacaactggggtaaagaggagcgtcaaagcgcgttcccgtttgagagcggcaaaccgttcaagattcaggtgctggttgaagcggaccactttaaagtggcggttaacgatgcgcacctgctgcaatataaccaccgtatgaagaacctgcgtgaaattagccaactgggcattagcggtgacattaccctgaccagcgcgaaccacgcgatgattggtggcagcggtggcagcttagaaaaatgttgtgctgcagctgatccccacgaatgttacgcaaaggtgtttgacgagtttaagccgctggttgaagaaccgcagaacctgatcaaacaaaactgcgagctgttcgaacagctgggcgagtacaagtttcaaaacgcgctgctggtgcgttataccaagaaagtgccgcaggttagcaccccgaccctggtggaagttagccgtaacctgggtaaagttggcagcaaatgctgcaaacatccggaggcgaagcgtatgccgtgcgcggaagactacctgagcgtggttctgaaccaactgtgcgtgctgcacgagaaaaccccggtgagcgatcgtgttaccaagtgctgcaccgaaagcctggttaaccgtcgtccgtgcttcagcgcgctggaagtggacgaaacctatgttccgaaagagtttaacgcggaaaccttcacctttcacgcggatatctgcaccctgagcgagaaggaacgtcagattaagaaacaaaccgcgctggttgagctggttaagcacaaaccgaaggcgaccaaagaacagctgaaggcggtgatggacgatttcgcggcgtttgttgagaaatgctgcaaggcgtaatgaaagctt

SEQ ID NO: 14 - protein, 355 aa, Artificial Sequence
  Synthetic Construct
MLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKAGGSGGSGAPAGPLIVPYNLPLPGGVVPRMLITILGTVKPNANRIALDFQRGNDVAFHFNPRFNENNRRVIVCNTKLDNNWGREERQSVFPFESGKPFKIQVLVEPDHFKVAVNDAHLLQYNHRVKKLNEISKLGISGDIDLTSASYTMI

SEQ ID NO: 15 - protein, 355 aa, Artificial Sequence
  Synthetic Construct
MAKCCKEVFAAFDDMVAKLQEKTAKPKHKVLEVLATQKKIQREKESLTCIDAHFTFTEANFEKPVYTEDVELASFCPRRNVLSETCCKTVRDSVPTKEHLVCLQNLVVSLYDEACPMRKAEPHKCCKSGVKGLNRSVEVLTPTSVQPVKKTYRVLLANQFKYEGLQEFLECNQKILNQPEEVLPKFEDFVKAYCEHPDAAACCKELGGSGGSGAPAGPLIVPYNLPLPGGVVPRMLITILGTVKPNANRIALDFQRGNDVAFHFNPRFNENNRRVIVCNTKLDNNWGREERQSVFPFESGKPFKIQVLVEPDHFKVAVNDAHLLQYNHRVKKLNEISKLGISGDIDLTSASYTMI

SEQ ID NO: 16 - protein, 355 aa, Artificial Sequence
  Synthetic Construct
MGAPAGPLIVPYNLPLPGGVVPRMLITILGTVKPNANRIALDFQRGNDVAFHFNPRFNENNRRVIVCNTKLDNNWGREERQSVFPFESGKPFKIQVLVEPDHFKVAVNDAHLLQYNHRVKKLNEISKLGISGDIDLTSASYTMIGGSGGSAKCCKEVFAAFDDMVAKLQEKTAKPKHKVLEVLATQKKIQREKESLTCIDAHFTFTEANFEKPVYTEDVELASFCPRRNVLSETCCKTVRDSVPTKEHLVCLQNLVVSLYDEACPMRKAEPHKCCKSGVKGLNRSVEVLTPTSVQPVKKTYRVLLANQFKYEGLQEFLECNQKILNQPEEVLPKFEDFVKAYCEHPDAAACCKEL

(19) United States
(12) Patent Application Publication
(10) Pub. No.: US 2021/0353715 A1
(43) Pub. Date: Nov. 18, 2021

(54) METHODS AND COMPOSITIONS FOR PREVENTING, TREATING, AND REVERSING LIVER FIBROSIS

(71) Applicant: MandalMed, Inc., San Francisco, CA (US)

(72) Inventors: Constance M. JOHN, San Francisco, CA (US); Khalil MOUNZIH, San Francisco, CA (US)

(73) Assignee: MandalMed, Inc., San Francisco, CA (US)

(21) Appl. No.: 17/277,719

(22) PCT Filed: Sep. 19, 2019
(86) PCT No.: PCT/US2019/052020

§ 371 Date: Mar. 18, 2021

Related U.S. Application Data
(60) Provisional application No. 62/734,184, filed on Sep. 20, 2018.

(51) Int. Cl. A61K 38/17; A61P 1/16

(52) U.S. Cl. CPC A61K 38/1732; A61P 1/16

(57) ABSTRACT

Methods of reducing, treating, or reversing liver fibrosis in a subject in need thereof comprising administering Galectin-3C, are provided. Also provided are Galectin-3C fusion proteins with increased stability.

### CROSS-REFERENCE TO RELATED APPLICATIONS

This application claims the benefit of U.S. Prov. App. Ser. No. 62/734,184, filed on Sep. 20, 2019, the disclosure of which is hereby incorporated by reference in its entirety.

### STATEMENT REGARDING FEDERALLY SPONSORED RESEARCH

This invention was made with government support under grant number 1 R43 DK107285-01A1, awarded by the National Institute of Diabetes and Digestive and Kidney Diseases. The government has certain rights to the invention.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 701642000240SEQLIST.TXT, date recorded: Sep. 19, 2019, size: 28 KB)).

### TECHNICAL FIELD

The present invention relates to methods of reducing, treating, or reversing liver fibrosis in a subject in need thereof comprising administering Galectin-3C. The present invention also relates to Galectin-3C fusion proteins with increased stability.

### BACKGROUND OF THE INVENTION

Chronic liver disease affects approximately 3 million people in the United States and leads to more than 1.2 million hospitalizations and about 43,000 deaths each year. Almost all chronic liver diseases are characterized by fibrosis, which results from a cascade of events that includes infiltration of inflammatory cells, apoptosis of hepatocytes, proliferation of matrix-producing mesenchymal cells, and increased deposition of collagen and other extracellular matrix (ECM) components. Liver fibrosis can progress to cirrhosis and hepatic failure (1), for which the only treatment option is liver transplantation. While resolution of liver fibrosis has been shown in animal models and in patients after treatment and/or removal of a causative agent (2-5), there are no approved agents that act to prevent or reverse fibrosis.

Recent studies implicate galectin-3 as a causal factor in liver fibrosis. For example, galectin-3 deficient mice were protected from liver fibrosis from carbon tetrachloride exposure or bile duct ligation (BDL), whereas wild type mice were not, despite equivalent injury. Inhibition of galectin-3 expression with small interfering (si) RNA had a therapeutic effect in the carbon tetrachloride mouse model of acute liver disease (6). In the BDL mouse model of chronic liver disease, galectin-3 expression was induced in hepatic stellate cells (HSCs) that give rise to the fibrogenic myofibroblasts (7). Systemic and hepatic vein levels of galectin-3 were increased in patients with alcoholic liver cirrhosis and the levels were negatively correlated with liver function (8).

There is a need in the art for the development of inhibitors of galectin-3 for the treatment of chronic liver fibrosis. The present application addresses these and other needs.

The disclosures of all publications, patents, patent applications and published patent applications referred to herein are hereby incorporated herein by reference in their entirety.

### SUMMARY

Provided is a method of reducing liver fibrosis in a subject in need thereof, comprising administering a therapeutically effective amount of Galectin-3C (Gal-3C) to the subject. Also provided is a method of treating liver fibrosis in a subject in need thereof, comprising administering a therapeutically effective amount of Gal-3C to the subject. Also provided is a method of reversing liver fibrosis in a subject in need thereof, comprising administering a therapeutically effective amount of Gal-3C to the subject. In some embodiments, the Gal-3C is produced by culturing allogeneic or autologous host cell comprising a nucleic acid encoding the Gal-3C under conditions where the Gal-3C is expressed, and implanting the host cells to supply the Gal-3C in vivo, or harvesting the Gal-3C expressed by the host cell for parenteral administration. In some embodiments, the subject has elevated serum galectin-3. In some embodiments, the Gal-3C is fused to human serum albumin, or a domain thereof, to form a fusion protein. In some embodiments, the fusion protein comprises a domain of human serum albumin selected from the group consisting of domain I (1DHSA), domain II (2DHSA) or domain III (3DHSA). In some embodiments, the fusion protein comprises domain III (3DHSA) of human serum albumin. In some embodiments, the C-terminus of 3DHSA is fused to the N-terminus of Gal-3C. In some embodiments, the N-terminus of 3DHSA is fused to the N-terminus of Gal-3C. In some embodiments, the C-terminus of 3DHSA is fused to the C-terminus of Gal-3C. In some embodiments, the N-terminus of 3DHSA is fused to the C-terminus of Gal-3C. In some embodiments, the fusion protein has an increased serum half-life compared to Gal-3C. Also provided is a fusion protein comprising Gal-3C fused to human serum albumin, or a domain thereof, wherein the fusion protein has an increased serum half-life compared to Gal-3C. In some embodiments, the fusion protein comprises a domain of human serum albumin selected from the group consisting of domain I (1DHSA), domain II (2DHSA) or domain III (3DHSA). In some embodiments, the fusion protein comprises domain III (3DHSA) of human serum albumin. In some embodiments, the C-terminus of 3DHSA is fused to the N-terminus of Gal-3C. In some embodiments, the N-terminus of 3DHSA is fused to the N-terminus of Gal-3C. In some embodiments, the C-terminus of 3DHSA is fused to the C-terminus of Gal-3C. In some embodiments, the N-terminus of 3DHSA is fused to the C-terminus of Gal-3C. In some embodiments, the subject is a human. In some embodiments the Gal-3C is human Gal-3C.

### BRIEF DESCRIPTION OF THE DRAWINGS

FIGS. 1A and 1B. Gal-3C treatment of PMA-activated human THP-1 monocytic cells polarized to the M2 phenotype by exposure to IL-4 inhibited the phenotypic expression of arginase (FIG. 1A) and IL-10 (FIG. 1B). Error bars=standard error of the mean.

FIG. 2A-2C. SDS-PAGE analysis of fused protein 3DHSA-Gal-3C mouse with E. coli expression by construct pET-30a(+) and 3 different (FIG. 2A, FIG. 2B, FIG. 2C) conditions. The fusion protein has the C-terminus of the 3^{rd} domain of human serum albumin (3DHSA) linked to the N-terminus of Gal-3C.

FIG. 3A-3C. SDS-PAGE analysis of fused protein mouse Gal-3C-3DHSA with E. coli expression by construct pET-30a(+) and 3 different (FIG. 3A, FIG. 3B, FIG. 3C) conditions. The fusion protein has the N-terminus of 3DHSA linked to the C-terminus of Gal-3C.

### DETAILED DESCRIPTION OF THE INVENTION

### Definitions

For purposes of interpreting this specification, the following definitions will apply and whenever appropriate, terms used in the singular will also include the plural and vice versa. In the event that any definition set forth below conflicts with any document incorporated herein by reference, the definition set forth shall control.

As used herein, the singular form “a”, “an”, and “the” includes plural references unless indicated otherwise.

It is understood that aspects and embodiments of the invention described herein include “comprising,” “consisting,” and “consisting essentially of” aspects and embodiments.

For all compositions described herein, and all methods using a composition described herein, the compositions can either comprise the listed components or steps, or can “consist essentially of” the listed components or steps. When a composition is described as “consisting essentially of” the listed components, the composition contains the components listed, and may contain other components which do not substantially affect the condition being treated, but do not contain any other components which substantially affect the condition being treated other than those components expressly listed; or, if the composition does contain extra components other than those listed which substantially affect the condition being treated, the composition does not contain a sufficient concentration or amount of the extra components to substantially affect the condition being treated. When a method is described as “consisting essentially of” the listed steps, the method contains the steps listed, and may contain other steps that do not substantially affect the condition being treated, but the method does not contain any other steps which substantially affect the condition being treated other than those steps expressly listed. As a non-limiting specific example, when a composition is described as “consisting essentially of” a component, the composition may additionally contain any amount of pharmaceutically acceptable carriers, vehicles, or diluents and other such components which do not substantially affect the condition being treated.

An “effective amount” or “therapeutically effective amount” as used herein refers to an amount of therapeutic compound, administered to a subject, either as a single dose or as part of a series of doses, which is effective to produce or contribute to a desired therapeutic effect, either alone or in combination with another therapeutic modality. An effective amount may be given in one or more dosages.

The term “treating” as used herein, refers to retarding the progress of or lessening the symptoms of a condition, such as fibrosis. The term “treatment,” as used herein, refers to the act of treating a condition, such as fibrosis.

The term “preventing” as used herein, refers to delaying the onset of, reduce the frequency of symptoms, or reduce the severity of symptoms associated with a condition, such as fibrosis.

The term “reversing” as used herein refers to reversing the progress of a condition, such as fibrosis.

A “subject” as used herein refers to any animal classified as a mammal, including humans, domestic and farm animals, and zoo, sport, or pet animals, such as dogs, horses, rabbits, cattle, pigs, hamsters, gerbils, mice, ferrets, rats, cats, and the like. In some embodiments, the subject is human.

The term “about” as used herein refers to the usual error range for the respective value readily known to the skilled person in this technical field. Reference to “about” a value or parameter herein includes (and describes) embodiments that are directed to that value or parameter per se.

For any of the structural and functional characteristics described herein, methods of determining these characteristics are known in the art.

### Overview

As provided herein, Gal-3C N-terminally truncated variants (collectively referred to herein interchangeably as “Gal-3C” in the singular) are N-terminally truncated forms of full length galectin-3 that lack the amino-terminal domain but retain carbohydrate binding ability. Gal-3C acts as a dominant negative inhibitor of galectin-3 by preventing the oligomerization of galectin-3 and its cross-linking of carbohydrate-containing ligands on cell surfaces and in the ECM, e.g., the hepatic ECM.

Provided herein is a method of reducing liver fibrosis (e.g., reducing the rate of liver fibrosis or delaying liver fibrosis) in a subject in need thereof comprising administering a Gal-3C variant provided herein to the subject. Also provided herein are methods of inhibiting (such as preventing) liver fibrosis in a subject in need thereof, comprising administering Gal-3C to the subject. Also provided herein are methods of reversing liver fibrosis in a subject in need thereof comprising administering Gal-3C to the subject. In some embodiments, the liver fibrosis is the result of, e.g., disease of the liver (such as cirrhosis or hepatitis) or bile duct, or injury to the liver or bile duct.

Viral infections, including hepatitis B and C, are primary causes of liver fibrosis. Other causes include alcoholism, metabolic and autoimmune disorders, toxins/drugs, helminthic infection, iron or copper overload and primary biliary cirrhosis. Non-alcoholic steatohepatitis (NASH) is a major cause of fibrosis from increasing obesity.

Fibrosis in the liver occurs as part of the wound-healing process due to chronic liver injury. It is the outcome of almost all chronic liver diseases, and if untreated can progress to cirrhosis and hepatic failure, or can be healed. Cirrhosis results when the scarring becomes so extensive that liver function and liver blood flow become disrupted. Fibrosis is due to the greatly increased deposition of collagen and other ECM components. There are no effective therapies for advanced cirrhosis, so treatment relies on liver transplantation. Overall, there is a great unmet medical need for new treatments for liver fibrosis.

### Gal-3C

In the some embodiments, Gal-3C used in a method provided herein comprises the 136, 137, 138, 139, 140, 141, or 142 carboxy-terminal amino acid residues of full length galectin-3 that suffice for carbohydrate binding (93). In some embodiments, Gal-3C comprises the 143 carboxy-terminal amino acid residues of full-length galectin-3. In some embodiments, Gal-3C comprises the sequence provided in SEQ ID NO: 3.

(SEQ ID NO: 3)
GAPAGPLIVP YNLPLPGGVV PRMLITILGT VKPNANRIAL
DFQRGNDVAF HFNPRFNENN RRVIVCNTKL DNNWGREERQ
SVFPFESGKP FKIQVLVEPD HFKVAVNDAH LLQYNHRVKK
LNEISKLGIS GDIDITSASY TMI

In some embodiments, Gal-3C comprises a sequence having at least 70%, 75%, 80%, 85%, 90%, 95%, 96%, 97%, 98%, or 99% identity to SEQ ID NO: 3 and retains carbohydrate binding ability.

Gal-3C Variants Comprising Amino Acid Substitution(s)

It is understood that amino acids may be substituted on the basis of side chain bulk, charge and/or hydrophobicity. Amino acid residues are classified into four major groups: acidic, basic, neutral/non-polar, and neutral/polar. In some embodiments, an acidic amino acid may be substituted by another acidic amino acid. In some embodiments, a basic amino acid may be substituted by another basic amino acid. In some embodiments, neutral/non-polar amino acid may be substituted by another neutral/non-polar amino acid. In some embodiments, neutral/polar amino acid may be substituted by another neutral/polar amino acid.

Amino acid residues can be further classified as cyclic or non-cyclic, aromatic or non-aromatic with respect to their side chain groups these designations being commonplace to the skilled artisan. In some embodiments, the following exemplary or preferred substitutions can be made to the amino acid sequences presented herein.

| Original | Exemplary Conservative | Preferred Conservative |
|---|---|---|
| Residue | Substitution | Substitution |
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | Ser |
| Gln | Asn | Asn |
| Glu | Asp | Asp |
| Gly | Pro | Pro |
| His | Asn, Gln, Lys, Arg | Arg |
| Ile | Leu, Val, Met, Ala | Leu |
| Phe | Leu | Ile, Val |
| Ile | Met, Ala, Phe | |
| Lys | Arg, Gln, Asn | Arg |
| Met | Leu, Phe, Ile | Leu |
| Phe | Leu, Val, Ile, Ala | Leu |
| Pro | Gly | Gly |
| Ser | Thr | Thr |
| Thr | Ser | Tyr |
| Tyr | Trp, Phe, Thr, Ser | Phe |
| Val | Ile, Leu, Met, Phe | Leu, Ala |

In some embodiments, alanine scanning mutagenesis as described by (9) can be utilized to introduce mutations to make Gal-3C variants.

In some embodiments, phage display of protein or peptide libraries provides a methodology for the selection of Gal-3C variants with improved affinity, altered specificity, or improved stability (10).

In some embodiments, modifications of a Gal-3C sequence include conserved mutation substitutions of one or more amino acids occurring between position 201 and 230 (where the Gal-3C comprises the carboxy terminal 143 amino acids of SEQ ID NO: 1). Possible conserved mutation substitutions include, but are not limited to, the following, where the amino acid on the left is the original and the amino acid on the right is the substituted amino acid.

| | | |
|---|---|---|
| Val-202 | → | Ala |
| Val-204 | → | Ala |
| Glu-205 | → | Asp |
| Asp-207 | → | Glu |
| His-208 | → | Arg |
| Phe-209 | → | Leu |
| Val-211 | → | Ala |
| Ala-212 | → | Val |
| Asp-215 | → | Glu |
| Ala-216 | → | Val |
| His-217 | → | Arg |
| Tyr-221 | → | Phe |
| His-223 | → | Arg |
| Val-225 | → | Ala |
| Glu-230 | → | Asp |

In some embodiments, amino acid substitutions can be performed using a PCR-based site-directed mutagenesis kit.

In some embodiments, the Gal-3C variant comprises an Asp-207→Glu substitution mutation (i.e., D207E). In some embodiments, the Gal-3C variant comprises the amino acid sequence of SEQ ID NO: 7.

(SEQ ID NO: 7)
GAPAGPLIVP YNLPLPGGVV PRMLITILGT VKPNANRIAL
DFQRGNDVAF HFNPRFNENN RRVIVCNTKL DNNWGREERQ
SVFPFESGKP FKIQVLVEPE HFKVAVNDAH LLQYNHRVKK
LNEISKLGIS GDIDLTSASY TMI

In some embodiments, the Gal-3C variant comprises a sequence having at least 70%, 75%, 80%, 85%, 90%, 95%, 96%, 97%, 98%, or even 99% identity to SEQ ID NO: 7 and retains the requisite carbohydrate binding ability.

In some embodiments, the Gal-3C variant comprises a Val-225→Ala substitution mutation (i.e., V225A). In some embodiments, the Gal-3C variant comprises the amino acid sequence of SEQ ID NO: 8

(SEQ ID NO: 8)
GAPAGPLIVP YNLPLPGGVV PRMLITILGT VKPNANRIAL
DFQRGNDVAF HFNPRFNENN RRVIVCNTKL DNNWGREERQ
SVFPFESGKP FKIQVLVEPE HFKVAVNDAH LLQYNHRAKK
LNEISKLGIS GDIDLTSASY TMI

In some embodiments, the Gal-3C sequence comprises a sequence having at least 70%, 75%, 80%, 85%, 90%, 95%, 96%, 97%, 98%, or even 99% identity to SEQ ID NO: 8 and retains the requisite carbohydrate binding ability.

Other Galectin-3 Variants

Other modifications and variations of galectin-3 for use in the methods described herein are possible. For example, full-length galectin-3 may instead be truncated at the C-terminus, producing a variant comprising the N-terminal residues of the galectin-3. It has been shown that both the C-terminal amino acids of galectin-3 comprising the carbohydrate binding domain (as described in detail herein) and the N-terminal oligomerization domain of galectin-3 (amino acids 1 to 107) act as inhibitors of the bioactivity of galectin-3 to induce angiogenesis and cancer cell migration in vitro (11). Galectin-3 can be endocytosed by cells by both carbohydrate and non-carbohydrate dependent mechanisms, the latter involving the non-carbohydrate recognition domain on the N-terminal part of galectin-3 (12). Thus the two truncated inhibitory forms of galectin-3, the 1-107 amino acids comprising the N-terminal non-carbohydrate recognition protein binding domain, and the 108-250 amino acids comprising the C-terminal carbohydrate recognition domain of galectin-3 would be expected to have differing degrees of endocytosis depending on the cell type and could have differing subcellular distribution. Since galectin-3 has different bioactivity in the nucleus, cytoplasm, and ECM, a therapy for liver fibrosis utilizing the C-terminally truncated variants or both domains of galectin-3 could be advantageous. The C-terminally truncated variants could be produced by one of normal skill in the art by cloning using the previously described methods (11).

Gal-3C Fusion Polypeptides

In some embodiments, the Gal-3C is attached (e.g., at its N-terminus or its C-terminus) to a human serum albumin (HSA), a serum-binding protein or peptide, or an organic molecule, e.g., a polymer (e.g., a polyethylene glycol (PEG)), in order to improve the pharmacokinetic properties of the Gal-3C, e.g., increase serum half-life. HSA proteins, serum-binding proteins or peptides, and organic molecules such as a PEG that serve to increase the serum half-life of Gal-3C are described in detail further herein. In some embodiments, the Gal-3C is attached to a human serum albumin, or a domain thereof. In some embodiments, attachment of Gal-3C to human serum albumin, or a domain thereof, allows for less frequent administration to a subject compared to Gal-3C. In some embodiments, the attachment is a covalent linkage. In some embodiments, Gal-3C is attached to a domain of human serum albumin selected from the group consisting of domain I (1DHSA), domain II (2DHSA) or domain III (3DHSA). In some embodiments, Gal-3C is attached to domain III (3DHSA) of human serum albumin. In some embodiments, the C-terminus of 3DHSA is attached to the N-terminus of Gal-3C. In some embodiments, the N-terminus of 3DHSA is attached to the N-terminus of Gal-3C. In some embodiments, the C-terminus of 3DHSA is attached to the C-terminus of Gal-3C. In some embodiments, the N-terminus of 3DHSA is attached to the C-terminus of Gal-3C.

Fusion to serum albumins can improve the pharmacokinetics of protein pharmaceuticals, and Gal-3C may be joined with a serum albumin. Serum albumin is a globular protein that is the most abundant blood protein in mammals. Serum albumin is produced in the liver and constitutes about half of the blood serum proteins. It is monomeric and soluble in the blood. Some of the most crucial functions of serum albumin include transporting hormones, fatty acids, and other proteins in the body, buffering pH, and maintaining osmotic pressure needed for proper distribution of bodily fluids between blood vessels and body tissues. In some embodiments, Gal-3C is fused to a serum albumin. In preferred embodiments, serum albumin is human serum albumin (HSA). An HSA that can be fused to Gal-3C is generally known in the art. In some embodiments, the HSA includes amino acids 25-609 of the sequence of UniProt ID NO: P02768. In some embodiments, the HSA includes one or more amino acid substitutions (e.g., C34S and/or K573P), relative to amino acids 25-609 of the sequence of UniProt ID NO: P02768.

Binding to serum proteins can improve the pharmacokinetics of protein pharmaceuticals, and in particular Gal-3C may be fused with serum protein-binding peptides or proteins. In some embodiments, Gal-3C may be fused to an albumin-binding peptide that displays binding activity to serum albumin to increase the half-life of Gal-3C. Albumin-binding peptides that can be used in the methods and compositions described here are generally known in the art (13,14). In one embodiment, the albumin binding peptide includes the sequence DICLPRWGCLW (SEQ ID NO: 9). An albumin-binding peptide can be fused genetically to Gal-3C or attached to Gal-3C through chemical means, e.g., chemical conjugation. If desired, a spacer can be inserted between Gal-3C and the albumin-binding peptide to allow for additional structural and spatial flexibility of the fusion protein. Specific spacers and their amino acid sequences are described in detail further herein. In some embodiments, an albumin-binding peptide may be fused to the N- or C-terminus of Gal-3C, e.g., via peptide bond or chemical conjugation techniques. Without being bound to a theory, it is expected that fusion of an albumin-binding peptide to Gal-3C may lead to prolonged retention of the therapeutic protein through its binding to serum albumin.

In some embodiments, Gal-3C may be fused to a polymer, e.g., polyethylene glycol (PEG). The attachment of a polymer to a protein pharmaceutical can “mask” the protein pharmaceutical from the host's immune system (15). In addition, certain polymers, e.g., hydrophilic polymers, can also provide water solubility to hydrophobic proteins and drugs (16,17). Various polymers, such as PEG, polysialic acid chain (16) and PAS chain (18) are known in the art and can be used in the present invention. In some embodiments, a polymer, e.g., PEG, may be covalently attached to Gal-3C, either at the N- or C-terminus or at an internal location, using conventional chemical methods, e.g., chemical conjugation. In some embodiments, a polymer, e.g., PEG, may be covalently attached to a cysteine substitution or addition in Gal-3C. The addition of a cysteine residue in Gal-3C may be introduced using conventional techniques in the art, e.g., peptide synthesis, genetic modification, and/or molecular cloning. The polymer, e.g., PEG, may be attached to the cysteine residue using cysteine-maleimide conjugation well-known to one of skill in the art. The contents of the referenced publications are incorporated herein by reference in their entireties.

In addition to the embodiments described above, other half-life extension technologies are also available and may be used in the present invention to increase the serum half-life of Gal-3C. Half-life extension technologies include, but are not limited to, and EXTEN (19) and Albu tag (20). The contents of the referenced publications are incorporated herein by reference in their entireties.

Methods of Producing Gal-3C

In some embodiments, Gal-3C is produced by subjecting the full-length human galectin-3 protein comprising the amino acid sequence of SEQ ID NO: 1 to exhaustive collagenase digestion.

(SEQ ID NO: 1)
MADNFSLHDA LSGSGNPNPQ GWPGAWGNQP AGAGGYPGAS
YPGAYPGQAP PGAYPGQAPP GAYPGAPGAY PGAPAPGVYP
GPPSGPGAYP SSGQPSATGA YPATGPYGAP AGPLIVPYNL
PLPGGVVPRM LITILGTVKP NANRIALDFQ RGNDVAFHFN
PRFNENNRRV IVCNTKLDNN WGREERQSVF PFESGKPFKI
QVLVEPDHFK VAVNDAHLLQ YNHRVKKLNE ISKLGISGDI
DLTSASYTMI

In some embodiments, Gal-3C is produced by subjecting SEQ ID NO: 1 to cleavage, e.g., with prostate specific antigen.

In some embodiments, Gal-3C is derived from full length human, rat, mouse, swine, cow, horse, feline, or canine galectin-3. In some embodiments, Gal-3C is derived from the full length galectin-3, which is encoded by the human LGALS3 gene that is located on chromosome 14, locus q21-q22 with cDNA sequence (49).

In some embodiments, Gal-3C is produced by culturing a host cell (e.g., a prokaryotic host cell or a eukaryotic host cell, such as a yeast cell, insect cell, or a mammalian cell) comprising a nucleic acid encoding the galectin-3 under conditions where the galectin-3 variant is expressed, harvesting the galectin-3 expressed by the host cell, and subjecting the galectin-3 harvested from the host cell to enzymatic cleavage or digestion. In some embodiments, the galectin-3 is subject to exhaustive collagenase digestion. In some embodiments, the galactin-3 is subject to cleavage with prostate specific antigen. In some the Gal-3C variant is purified from the digestion reaction or cleavage reaction via affinity chromatography on lactosyl-sepharose.

In some embodiments, the nucleic acid encoding galectin-3 protein comprises the sequence of SEQ ID NO: 2 starting from the underlined ATG up to the underlined ATA.

(SEQ ID NO: 2)
AGCAGCCGTC CGGAGCCAGC CAACGAGCGG AAAATGGCAG
ACAATTTTTC GCTCCATGAT GCGTTATCTG GGTCTGGAAA
CCCAAACCCT CAAGGATGGC CTGGCGCATG GGGGAACCAG
CCTGCTGGGG CAGGGGGCTA CCCAGGGGCT TCCTATCCTG
GGGCCTACCC CGGGCAGGCA CCCCCAGGGG CTTATCCTGG
ACAGGCACCT CCAGGCGCCT ACCATGGAGC ACCTGGAGCT
TATCCCGGAG CACCTGCACC TGGAGTCTAC CCAGGGCCAC
CCAGCGGCCC TGGGGCCTAC CCATCTTCTG GACAGCCAAG
TGCCCCCGGA GCCTACCCTG CCACTGGCCC CTATGGCGCC
CCTGCTGGGC CACTGATTGT GCCTTATAAC CTGCCTTTGC
CTGGGGGAGT GGTGCCTCGC ATGCTCATAA CAATTCTGGG
CACGGTGAAG CCCAATGCAA ACAGAATTGC TTTAGATTTC
CAAAGAGGGA ATGATGTTGC CTTCCACTTT AACCCACGCT
TCAATGAGAA CAACAGGAGA GTCATTGTTT GCAATACAAA
GCTGGATAAT AACTGGGGAA GGGAAGAAAG ACAGTCGGTT
TTCCCATTTG AAAGTGGGAA ACCATTCAAA ATACAAGTAC
TGGTTGAACC TGACCACTTC AAGGTTGCAG TGAATGATGC
TCACTTGTTG CAGTACAATC ATCGGGTTAA AAAACTCAAT
GAAATCAGCA AACTGGGAAT TTCTGGTGAC ATAGACCTCA
CCAGTGCTTC ATATACCATG ATATAATCTG AAAGGGGCAG
ATTAAAAAAA AAAACGGA

In some embodiments, Gal-3C is produced by culturing a host cell (e.g., a prokaryotic host cell or a eukaryotic host cell, such as a yeast cell, insect cell, or a mammalian cell) comprising a nucleic acid encoding Gal-3C under conditions where the Gal-3C variant is expressed, harvesting the Gal-3C expressed by the host cell or implanting the genetically modified host cell.

In some embodiments, the Gal-3C encoded by the nucleic acid comprises the amino acid sequence of SEQ ID NO: 3. In some embodiments, the Gal-3C encoded by the nucleic acid comprises an amino acid sequence having at least 70%, 75%, 80%, 85%, 90%, 95%, 96%, 97%, 98%, or 99% identity to SEQ ID NO: 3.

In some embodiments, the Gal-3C encoded by the nucleic acid comprises the amino acid sequence of SEQ ID NO: 7. In some embodiments, the Gal-3C encoded by the nucleic acid comprises an amino acid sequence having at least 70%, 75%, 80%, 85%, 90%, 95%, 96%, 97%, 98%, or 99% identity to SEQ ID NO: 7.

In some embodiments, the Gal-3C encoded by the nucleic acid comprises the amino acid sequence of SEQ ID NO: 8. In some embodiments, the Gal-3C encoded by the nucleic acid comprises an amino acid sequence having at least 70%, 75%, 80%, 85%, 90%, 95%, 96%, 97%, 98%, or 99% identity to SEQ ID NO: 8.

In some embodiments, the Gal-3C is encoded by a nucleic acid comprising the nucleotide sequence of SEQ ID NO: 4.

(SEQ ID NO: 4)
ATGGGCGCCC CTGCTGGGCC ACTGATTGTG CCTTATAACC
TGCCTTTGCC TGGGGGAGTG GTGCCTCGCA TGCTCATAAC
AATTCTGGGC ACGGTGAAGC CCAATGCAAA CAGAATTGCT
TTAGATTTCC AAAGAGGGAA TGATGTTGCC TTCCACTTTA
ACCCACGCTT CAATGAGAAC AACAGGAGAG TCATTGTTTG
CAATACAAAG CTGGATAATA ACTGGGGAAG GGAAGAAAGA
CAGTCGGTTT TCCCATTTGA AAGTGGGAAA CCATTCAAAA
TACAAGTACT GGTTGAACCT GACCACTTCA AGGTTGCAGT
GAATGATGCT CACTTGTTGC AGTACAATCA TCGGGTTAAA
AAACTCAATG AAATCAGCAA ACTGGGAATT TCTGGTGACA
TAGACCTCAC CAGTGCTTCA TATACCATGA TA

In some embodiments, nucleic acid has been optimized for expression in Escherichia coli. The parameters that may be optimized include, e.g., codon usage bias, GC content, CpG dinucleotide content, secondary structure of mRNA, cryptic splicing sites, premature PolyA sites, internal chi sites and ribosomal binding sites, negative CpG islands, RNA instability motif, repeat sequences (direct repeat, reverse repeat, and Dyad repeat), and restriction sites that may interfere with cloning. In some embodiments, the length of an optimized human galectin-3 sequence from which Gal-3C is derived is 753 base pairs with GC %: 57.87. An example of the optimized gene is shown in SEQ ID NO: 5.

(SEQ ID NO: 5)
ATGGCAGATA ACTTCTCGCT GCATGACGCA CTGTCGGGCT
CGGGTAATCC GAATCCGCAG GGCTGGCCGG GCGCTTGGGG
TAATCAACCG GCAGGTGCCG GCGGTTATCC GGGTGCTTCT
TATCCGGGCG CATACCCGGG TCAGGCTCCG CCGGGTGCAT
ACCCGGGTCA AGCACCGCCG GGTGCATATC ATGGTGCACC
GGGTGCTTAC CCGGGTGCAC CGGCTCCGGG TGTGTATCCG
GGTCCGCCGT CAGGCCCGGG TGCCTACCCG AGCTCTGGTC
AGCCGTCGGC ACCGGGTGCA TATCCGGCAA CGGGTCCGTA
CGGTGCACCG GCAGGTCCGC TGATTGTTCC GTATAACCTG
CCGCTGCCGG GCGGTGTGGT TCCGCGTATG CTGATTACCA
TCCTGGGCAC GGTCAAACCG AACGCTAATC GTATTGCGCT
GGATTTTCAA CGCGGTAACG ACGTGGCGTT TCATTTCAAC
CCGCGCTTCA ATGAAAACAA TCGTCGCGTC ATCGTGTGCA
ATACCAAACT GGATAACAAT TGGGGCCGTG AAGAACGCCA
GAGTGTTTTT CCGTTCGAAT CCGGTAAACC GTTTAAAATC
CAAGTTCTGG TCGAACCGGA TCACTTCAAA GTGGCCGTTA
ATGACGCACA TCTGCTGCAG TATAACCACC GTGTCAAAAA
ACTGAATGAA ATTAGTAAAC TGGGCATTTC TGGCGACATT
GACCTGACCT CGGCGTCCTA CACGATGATT TAA

In some embodiments, the nucleic acid comprises the sequence of SEQ ID NO: 6.

(SEQ ID NO: 6)
ATGGGTGCAC CGGCAGGTCC GCTGATTGTT CCGTATAACC
TGCCGCTGCC GGGCGGTGTG GTTCCGCGTA TGCTGATTAC
CATCCTGGGC ACGGTCAAAC CGAACGCTAA TCGTATTGCG
CTGGATTTTC AACGCGGTAA CGACGTGGCG TTTCATTTCA
ACCCGCGCTT CAATGAAAAC AATCGTCGCG TCATCGTGTG
CAATACCAAA CTGGATAACA ATTGGGGCCG TGAAGAACGC
CAGAGTGTTT TTCCGTTCGA ATCCGGTAAA CCGTTTAAAA
TCCAAGTTCT GGTCGAACCG GATCACTTCA AAGTGGCCGT
TAATGACGCA CATCTGCTGC AGTATAACCA CCGTGTCAAA
AAACTGAATG AAATTAGTAA ACTGGGCATT TCTGGCGACA
TTGACCTGAC CTCGGCGTCC TACACGATGA TT

### Pharmaceutical Compositions

The Gal-3C variants can be formulated with suitable carriers or excipients so that they are suitable for administration for the treatment, prevention, or reversal of liver fibrosis. Suitable formulations of the Gal-3C variants are obtained by mixing a Gal-3C variant having the desired degree of purity with optional pharmaceutically acceptable carriers, excipients or stabilizers (21) in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propylparaben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as olyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). Exemplary formulations are described in WO98/56418, expressly incorporated herein by reference. Lyophilized formulations adapted for subcutaneous administration are described in WO97/04801. Such lyophilized formulations may be reconstituted with a suitable diluent to a high protein concentration and the reconstituted formulation may be administered subcutaneously to the mammal to be treated herein.

The formulation herein may also contain more than one active compound as necessary for the particular indication being treated, preferably those with complementary activities that do not adversely affect each other. For example, it may be desirable to further provide an anti-neoplastic agent, a growth inhibitory agent, a cytotoxic agent, or a chemotherapeutic agent. Such molecules are suitably present in combination in amounts that are effective for the purpose intended. The effective amount of such other agents depends on the amount of the Gal-3C variant present in the formulation, the type of disease or disorder or treatment, and other factors discussed above. These are generally used in the same dosages and with administration routes as described herein or about from 1 to 99% of the heretofore employed dosages. The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980). Sustained-release preparations may be prepared. Suitable examples of sustained release preparations include semi-permeable matrices of solid hydrophobic polymers containing the antagonist, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and. ethyl-L-glutamate, non-degradable ethylene-vinyl, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

Lipofectins or liposomes can be used to deliver the Gal-3C variants provided herein invention into cells. A Gal-3C variant can also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles, and nanocapsules) or in macroemulsions. Such techniques are disclosed in Remington's PHARMACEUTICAL SCIENCES, supra.

Sustained-release preparations can be prepared. Suitable examples of sustained-release preparations include semipermeable matrices of solid hydrophobic polymers containing the Gal-3C variant, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydro gels release proteins for shorter time periods. When encapsulated antibodies remain in the body for a long time, they can denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization can be achieved by modifying sulfhydryl residues, lyophilizing from acidic solutions, controlling moisture content, using appropriate additives, and developing specific polymer matrix compositions.

The formulations to be used for in vivo administration must be sterile. This is readily accomplished by, e.g., filtration through sterile filtration membranes.

Dosing

In some embodiments, for in vivo administration of a Gal-3C variant described herein, dosage amounts for humans based on use of the appropriate body surface area conversion factor (BSA-CF) may vary from about 0.25 mg/kg/day to about 0.35 mg/kg/day, from about 0.25 mg/kg/day to about 0.45 mg/kg/day, from about 0.15 mg/kg/day to about 0.35 mg/kg/day, from about 0.2 mg/kg/day to about 0.3 mg/kg/day, from about 0.2 mg/kg/day to about 0.4 mg/kg/day, from about 0.3 mg/kg/day to about 0.5 mg/kg/day, from about 0.15 mg/kg/day to about 0.45 mg/kg/day, or even from about 0.4 mg/kg/day to about 0.9 mg/kg/day depending partly upon differences in exposure due to the route of administration, age, gender, and other factors. In some embodiments, the dosage for humans is 0.25 mg/kg/day to 0.35 mg/kg/day. In some embodiments, the dosage for humans is 0.20 mg/kg/day to 0.30 mg/kg/day. In some embodiments, the dosage for humans is 0.25 mg/kg/day to 0.40 mg/kg/day. In some embodiments, the dosage for humans is 0.25 mg/kg/day to 0.45 mg/kg/day. In some embodiments, the dosage for humans is about 0.25 mg/kg/day. In some embodiments, the dosage for humans is about 0.30 mg/kg/day. In some embodiments, the dosage for humans is about 0.35 mg/kg/day. In some embodiments, the dosage for humans is 0.25 mg/kg/day. In some embodiments, the dosage for humans is 0.30 mg/kg/day. In some embodiments, the dosage for humans is 0.35 mg/kg/day. For repeated administrations over several days or longer, depending on the severity of the disease or disorder to be treated, the treatment is sustained until a desired response is achieved.

Variable dosage regimens may be useful, depending on the route of administration, pharmacokinetics of the Gal-3C variant in humans, and the desired exposure levels and duration of exposure desired. Dosing an individual continuously using a pump for systemic delivery or from one to twenty-one times a week is contemplated herein. In certain embodiments, dosing frequency is three times per day, twice per day, once per day, once every other day, once weekly, once every two weeks, once every four weeks, or longer.

It is noted that use of Gal-3C variant in the methods provided herein uses doses significantly higher than those used for the treatment of cancer (e.g. see U.S. Pat. No. 9,272,014).

Administration

In some embodiments, the Gal-3C variant is administered intravenously, intramuscularly, subcutaneously, topically, transdermally, intraperitoneally, via secretion by implanted genetically-modified cells, by inhalation, intrathecally, intraventricularly, or intranasally. An effective amount of the Gal-3C variant may be administered for the treatment or prevention of fibrosis/fibrotic remodeling/liver cirrhosis. The appropriate dosage of the Gal-3C variant may be determined based on the extent of the liver fibrosis to be treated and the particular Gal-3C variant, the clinical condition of the individual, the individual's clinical history and response to the treatment, and the discretion of the attending physician.

In some embodiments, for example in a hospitalized patient, a solution comprising a Gal-3C variant may be continuously delivered intravenously (IV) by infusion using traditional IV bags in phosphate-buffered saline or normal saline. For ambulatory patients the Gal-3C variant may be delivered IV with non-electronic elastomeric external (Infusor; Baxter Corporation) pumps such as are used for chemotherapeutic and anesthetic agents. Baxter's “Infusor” elastomeric pumps provide duration infusion times from 12 hours to 7 days, are available with 7 different volumes varying from 48-272 milliliters, and have no cords, batteries or IV poles. These lightweight pumps do not require programming and have silent operation.

Using the Seven-Day Infusor (2C1082KP), continuous infusion of therapeutic agents for periods or more than 9 months have been used clinically (83). The Seven Day Infusor has a 95 milliliter volume and can be used for 7-day continuous IV delivery, i.e., of approximately 12 milliliter per day or 0.5 milliliter per hour. Thus, to deliver 20 milligrams of a Gal-3C variant per day to a 60 kilogram person for 7 days, a solution of 1.67 milligrams a Gal-3C variant with 4 milligrams lactose per milliliter of PBS could be used in the Seven Day Infusor. To deliver 30 milligrams per day to a 90-kilogram person for 7 days, a solution of 2.5 milligrams of a Gal-3C variant with 5 milligrams of lactose per milliliter of phosphate buffered saline could be used in the Seven-Day Infusor.

Alternatively, the Gal-3C variant can be delivered intradermally using the Hollow Microstructured Transdermal System (hTMS; 3M Company) for microneedle-based administration from 2-4 times daily. This is an integrated reservoir and infusion device that is designed for rapid delivery of liquid formulations of small molecules and biologics such as proteins and peptides. The 3M hMTS enables delivery of 0.5 to 2.0 milliliters over a few minutes (84). The single-use delivery system is formed by a 1 square centimeter array molded out of medical grade polymer and is designed for self-administration. Using the hMTS an approximately 20-milligram daily dose of the Gal-3C variant could be administered as 3 divided doses in 24 hours. Each dose would be 6.75 milligrams of the Gal-3C variant in a solution of 1.5 milliliters (4.5 milligrams per milliliter of the Gal-3C variant) with 5 milligrams lactose per milliliter in phosphate-buffered saline. For a 90-kilogram person, an approximately 30 milligram daily dose could be administered as 3 divided doses in 24 hours. Each dose would be 10 milligrams of the Gal-3C variant contained in a solution of 2 milliliters (5 milligrams per milliliter of the Gal-3C variant) with 10 milligrams lactose per milliliter in phosphate-buffered saline.

In another alternative, Gal-3C can be formulated to achieve a high concentration of soluble protein (50-200 mg/ml) and administered by subcutaneous injection in one daily injection. Progress of the therapy can be monitored by conventional techniques and assays. The dosing regimen, including the variant of the Gal-3C variant administered, can vary over time independently of the dose used.

Combination Therapies

In some embodiments, the Gal-3C variant is administered in combination with a second drug, such as a drug for treating liver disease, including, without limitation, e.g., cholic acid, Cholbam, neomycin, paromomycin, Humatin, Neo-Tab, Neo-Fradin, Paromycin, ursodiol, Actigall, Urso, colchicine, Urso Forte, azathioprine, obeticholic acid, and Ocaliva.

It is to be understood that the terminology employed herein is used for the purpose of describing particular embodiments only and is not intended to be limiting. It will be readily apparent to those skilled in the art that other suitable modifications and adaptations of the methods and compositions described herein may be made using suitable equivalents without departing from the scope of the embodiments disclosed herein. The following examples are for illustrative purposes. These are intended to show certain aspects and embodiments of the present invention but are not intended to limit the invention in any manner. All patents, patent applications and references described herein are incorporated in their entireties for all purposes.

### Examples

### Example 1: Effects of Gal-3C on the Profibrotic Activity of Primary Human Hepatic Stellate Cells In Vitro

In chronic liver fibrosis, hepatic stellate cells (HSCs) become activated and transdifferentiate to myofibroblast-like cells leading to accumulation of ECM. The activation of HSCs is thought to be due to inflammation mediated by immune cells, primarily macrophages. Resident macrophages (Kupffer cells) are thought to play a role in acute liver injury, but both acute and chronic liver injuries are characterized by a dramatic expansion of the hepatic macrophage population owing to the massive infiltration of monocytes into the liver (22).

We have shown that Gal-3C demonstrates antifibrotic activity when administered for only 1 week beginning on day 4 after injury activity in a rat ischemia-reperfusion model (I/R) of myocardial infarction (MI) as reported (23). However, liver fibrosis is significantly different from fibrosis of the myocardium post-MI. The heart, compared to other organs (and especially the liver), has very limited capacity for regeneration. The process of repair in the heart consists of removal of necrotic cardiomyocytes followed by replacement with fibrotic tissue that can preserve the structural integrity of the heart. However, the scar tissue is neither conductive nor contractile and, thus, the functionality of the heart is reduced. In MI, the lack of oxygen leads to the most death of cells in a discrete area of the myocardium based on where the lack of oxygen caused death to cardiomyocytes. By contrast, liver fibrosis (e.g., due to disease or injury to the liver or bile duct) is normally not localized.

To determine the effects of Gal-3C on HSC migration and collagen I secretion, the uptake of Gal-3C and galectin-3 in HSCs and galectin-3 expression levels in HSCs are analyzed. The assays used in the analyses are performed as follows.

Cell Culture.

Human cells are handled with BBP precautions. Primary human HSCs are obtained from ScienCell (Carlsbad, Calif.) and cultured in defined media as described in Das et al. (24). Culturing primary human HSCs on plastic culture ware for a few days stimulates differentiation of primary HSCs to a myofibroblast phenotype (25).

Assessment of Cellular Expression and Uptake of Galectin-3 and Gal-3C.

Galectin-3 and Gal-3C are labeled with fluorescein isothiocyanate (FITC) to permit the analysis of uptake, localization, and cell surface binding of each protein by HSCs and by human macrophages using confocal microscopy and flow cytometry as described in Feizi et al. (26). To detect endogenous galectin-3, cells are fixed and permeabilized with 4% PFA and 0.1% saponin prior to staining with allophycocyanin (APC)-conjugated goat anti-galectin-3 pAb (R&D Systems) at room temperature. Fixed cells are then analyzed via FACS and visualized via microscopy. Control experiments performed with cells incubated with labeled galectin-3 or Gal-3C in the presence of carbohydrate inhibitors of uptake are performed as described (12). Galectin-3 expression levels and levels of galectin-3 secretion by cells of varying confluency (24, 45, 95%) will are compared by performing Western blots and ELISA on cell lysates and supernatants, as described in Mirandola et al (27).

Migration Assays.

Cell migration assays are performed in 24-well, 6.5-mm diameter FluoroBlok (BD Biosciences) inserts (5-μm pore size) in transwell plates as described (28,29). Briefly, serum-starved HSCs, M0, M1, or M2 cells (150,000) in media with or without Gal-3C (0.5-20 μg/ml), and with or without pre-incubation with Gal-3C are loaded in upper chambers. For HSCs, media in lower chambers contains platelet-derived growth factor bb (PDGF-bb) (10 ng/ml) or TGFβ₁ (5 ng/ml) as described in Atorrasagasti et al. and Coffelt et al. (28,29). For macrophages media in lower chambers contains CCL2 (MCP-1) (2 ng/ml) or CCLS (1 ng/ml) as described in Vogel et al. (30). Control experiments are performed using exogenous galectin-3 (0.5-10 μg/ml), lactose (30 mM) and sucrose (30 mM) as described in Mirandola et al. (27). After incubation for 12-16 h, migratory cells on the bottom are stained and counted in 4 fields/filter. As a positive control, Gal-3C is used to inhibit migration of multiple myeloma cells as reported previously in Mirandola et al. (27). Experiments is performed in triplicate.

The following experiments are performed to further assess the effects of Gal-3C on HSC migration: Actin cytoskeleton reorganization and lamellipodia formation is analyzed with fluorescently-labeled phalloidin that binds F-actin, and Abs to human FAK and FAK-PY397 as described (28,31).

Real-Time (RT) PCR and ELISA.

Total RNA is isolated from HSCs and macrophages after incubation with or without Gal-3C (0.5-20 μg/ml) with an RNAeasy kit. cDNA is prepared (SuperScript VILO™ cDNA Synthesis Kit; Life Tech/Thermo), and genetic expression of procollagen-1α (I), α-SMA, in HSCs and for expression of IFN-γ, TNF-α, IL-1β, and IL-6 (for M1), and IL4, IL-10, and TGF-β (for M2) by all cells is quantified using SYBR Premix Ex Taq 3 and primers as described (32). Additionally, supernatants are harvested from all cells after culture with or without Gal-3C and frozen at 70° C. and then used for ELISA (eBiocience/Thermo Fisher) to quantify levels of IL-4, IL-10, IL-6, and TNFα protein.

### Example 2: Effects of Gal-3C on Profibrotic Macrophage Polarization

As M1 (classic/proinflammatory) macrophages and M2 (alternative/anti-inflammatory) macrophages, have been shown to play important roles in liver fibrosis (22,33,34) the effects of Gal-3C on the polarization and migration of M1 macrophages, M2 macrophages, and PMA-activated human THP-1 monocytic cells were analyzed.

Macrophages regulate inflammation and angiogenesis in wound healing and macrophages adopt classically activated (M1) proinflammatory or alternatively activated (M2) wound healing phenotypes in response to environmental signals(35). The M1 and M2 macrophages express distinct molecular markers. M2 polarization characteristically upregulates expression of the mannose receptor, dectin-1, Ym1, Fizz1, TGF-β, arginase-1, and IL-10. Galectin-3 is expressed by macrophages; its levels increase more than 5-fold in M1 macrophages and 15-fold in M2 macrophages (36). Also, galectin-3 activates M2 macrophages (37).

We determined whether Gal-3C treatment would inhibit activation of M2 macrophages that are implicated in the differentiation of myofibroblasts and induction of excessive fibrosis using a human monocytic model cell line. Increased expression of arginase-1 and interleukin (IL)-10 were used as molecular markers for the M2 phenotype.

Human THP-1 monocytic cells (ATCC, Manassas, Va., USA) were cultured at a density of 3-8×105 cells/mL in growth media (RPMI-1640 with 2 mM L-glutamine and 10% fetal bovine serum (Gibco/Thermofisher, Pittsburgh, Pa., USA) in a humidified cell culture incubator in 5% CO₂ at 37° C. Cells were passaged twice a week and used within 10 passages.

Cells were plated at 3×10⁵ cells/well in 6-well cell culture plates for analysis of arginase, and at 10,000 cells/well in 96-well plates for analysis of IL-10. Cells were cultured in growth media with 50 ng/mL phorbal myristate acetate (PMA; Sigma-Aldrich, St. Louis, Mo., USA) for 24 h. Then media containing non-adherent cells was removed and growth media containing 50 ng/mL phorbol myristate acetate (PMA) and with or without 20 ng/mL IL-4 (Sigma-Aldrich) was added to wells and adherent cells were cultured for another 72 h. Next, cell culture media was removed, and wells were washed with PBS and serum free RPMI 1640 media with and without Gal-3C (5 μg/mL) was added to wells, and cells were cultured for another 72 h. Finally, after centrifugation of plates to pellet cell debris, media was collected from wells and stored frozen at −80° C. for later analysis.

For measurement of arginase activity, the Quantichrome Arginase Assay Kit (Bioassay Systems, Hayward, Calif., USA) was used according to the instructions of the manufacturer. For quantification of the levels of IL-10, the human IL-10 ELISA kit (Abcam, Cambridge, Mass., USA) was used according to the instructions of the manufacturer. Plates were analyzed on a Thermomax or a Spectramax Plus microplate reader (Molecular Devices, San Jose, Calif., USA).

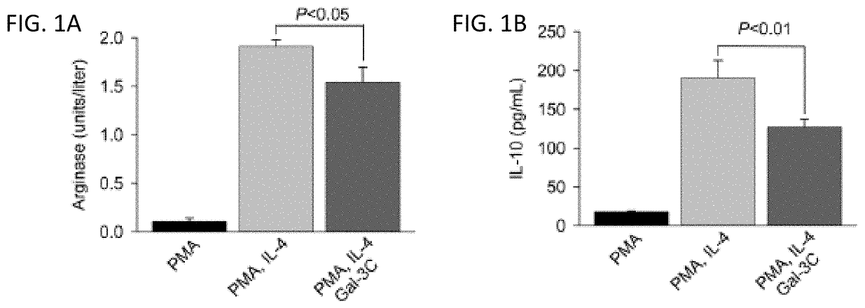

The results (FIGS. 1A and 1B) showed that treatment of PMA-treated THP-1 cells with IL-4 significantly increased levels of arginase and IL-10 that are characteristically expressed by M2 polarized macrophages, which promote fibrosis as part of the wound-healing response Gal-3C treatment reduced levels of arginase and IL-10 that are characteristically expressed by activated M2-polarized macrophage cells. These results support the potential therapeutic efficacy of Gal-3C in reducing liver fibrosis.

Testing in a format with primary cells, human peripheral blood CD14⁺ monocytes from StemCell Technologies (Vancouver, BC) was used to produce macrophages. The primary human monocytes (StemCell) were cultured (1×10⁶ cells/ml) in DMEM with 5% human serum for 5-7 days. Differentiation into adherent macrophages was confirmed by analysis of CD68 expression by flow cytometry. M1-type macrophages were generated by culture with IFNγ (1,000 U/ml), and then with E. coli LPS (10 ng/ml; 026:B6; Sigma-Aldrich). M2 macrophages were generated by culture in medium with IL-4 (10 ng/ml) and M-CSF (30)0 ng/ml). M0 macrophages were cultured in medium without additives. Alternatively, Monocyte Attachment, M1 and M2 Macrophage Generation Media from Promocell (Heidelberg, Del.) was used. Intracellular and cell-surface galectin-3 levels increase with M2 polarization (36) and galectin-3 can enhance M2 infiltration Effects of Gal-3C (0.5-20 μg/ml) on polarized macrophages was determined after incubation as described above. Control experiments using exogenous galectin-3 (0.5-10 μg/ml), lactose (30 mM), and sucrose (30 mM) were performed as described (27). Cytokine expression was analyzed by real-time (RT) PCR and ELISA.

### Example 3: Effects of Gal-3C in Mouse Models of Liver Fibrosis

Bile-duct ligation (BDL) and carbon tetrachloride (CCl₄) mouse models are used assess the effects of the murine analog of Gal-3C on liver fibrosis in immunocompetent C57/BL6 mice, as described (7,32,38). In the BDL model, disruption of bile secretion leads to progressive damage (7,39,40) and induces cholestatic fibrosis. BDL stimulates proliferation of biliary epithelium and oval cells resulting in proliferation of the intralobular bile ductules and portal inflammation (41,42). The CCl₄-induced toxic model causes inflammation and oxidative stress that spreads to the vasculature of the hepatic sinusoid and induces more extensive hepatocellular injury. Both models are considered highly reproducible (see (43)). Prevention and treatment of liver fibrosis is analyzed in both the BDL and CCl₄ models, and reversal of liver fibrosis is analyzed in the CCl₄ model as previously reported (38,44,45).

Production of Murine Analog of Gal-3C.

The murine Lgals3 gene is cloned and inserted into an E. coli PET28a+ expression vector. Two 5-liter batches of the PET28a+ E. coli are fermented using the BioFlo 120 fermenter to produce murine galectin-3. Bacterial pellets are lysed with B-Per reagent (Thermo Fisher), and the protein is purified on a lactosyl-sepharose column. Murine Gal-3C is produced by collagenase digestion of the murine galectin-3, and the Gal-3C is purified by lactosyl-sepharose chromatography as described in John et al. (46). The sequence of murine Gal-3C is confirmed by LC-MS/MS at Applied Biomics (Hayward, Calif.).

BDL Mouse Model of Chronic Liver Disease.

BDL is performed in two locations on pentobarbital-anesthetized mice as described (42,47). Mice in the control group undergo sham operation, i.e., laparotomy with exposure but not ligation. To assess whether murine Gal-3C prevents liver fibrosis, murine Gal-3C is delivered continuously intraperitoneally by osmotic pump for 21 days, beginning the day before BDL. To assess whether murine Gal-3C treats established liver fibrosis, murine Gal-3C is delivered for 14 days beginning day 7 post-BDL. Animals are sacrificed on day 21 post-BDL (see Table 1 below).

TABLE 1
Bile Duct Ligation (BDL) Experiments

| | | Prevention Grps | Treatment Groups | | | |
|---|---|---|---|---|---|---|
| Surgery | N | Gal-3C D 1-21 | Gal-3C D 8-21 | Weekly Analysis | Endpoints | Total Mice |
| Sham | 4 | Vehicle, Gal-3C | Vehicle, Gal-3C | ALT, AST, ALP, | qPCR for procollagen-1α (I), α-SMA, TNF-α, | 16 (4 groups × |
| | | | | bilirubin, galectin-3 | and β-actin in liver; Picosirius staining of | 4/group) |
| BDL | 14 | Vehicle, Gal-3C | Vehicle, Gal-3C | levels; body weight | collagen & immunochistochemistry (IHC) of | 56 (4 groups × |
| | | | | | α-SMA in liver, liver weight | 14/group) |

Carbon Tetrachloride (CCl₄) Mouse Model of Chronic Liver Disease.

Mice are injected IP with 1 μl/g CCl₄ in olive oil (1:3) or olive alone twice weekly for 8 weeks as described (6). To assess whether murine Gal-3C prevents liver fibrosis, murine Gal-3C is continuous delivered over a period of 8 weeks, beginning before the first injection of CCl₄. To assess whether murine Gal-3C treats established liver fibrosis, mice are treated with murine Gal-3C for 4 weeks, beginning 4 weeks after the first CCl₄ injection. To assess whether murine Gal-3C reverses liver fibrosis, murine Gal-3C is administered to mice for 1 week, beginning 8 weeks after the first CCl₄ injection. The mice in which the prevention of liver fibrosis and the treatment of liver fibrosis is being assessed are euthanized on day 56 (8 weeks) after the first CCl₄ injection. The mice in which the reversal of liver fibrosis is being assessed are euthanized on day 77, i.e., 2 weeks after ending Gal-3C treatment. See Table 2 below.

TABLE 2
Carbon Tetrachloride (CCL) Experiments

| | | Prevention Grps | Treatment Grps | Reversal Grps | | | |
|---|---|---|---|---|---|---|---|
| Injection | N | Gal-3C D1-56 | Gal-3C D 29-56 | Gal-3C 57-63 | Weekly Analysis | Endpoints | Mice |
| Olive oil | 4 | Vehicle, Gal-3C | Vehicle, Gal-3C | Vehicle, Gal-3C | ALT, AST, ALP, | qPCR for procollagen-1α (I), | 24 (6 grps × |
| | | | | | bilirubin, galectin-3 | α-SMA, TNF-α, and | 4/grp) |
| Olive oil | 14 | Vehicle, Gal-3C | Vehicle, Gal-3C | Vehicle, Gal-3C | levels; body weight | β-actin in liver, Picosirius | 84 (6 grps × |
| with CCl₄ | | | | | | staining of collagen & IHC** of | 14/grp) |
| | | | | | | α-SMA in liver, liver weight | |

Administration of Gal-3C.

In one method, to help ensure that therapeutic levels of murine Gal-3C are being maintained, murine Gal-3C is administered (IP) using an IP catheter attached to an osmotic mini-pump (Alzet, Durect Corp.) implanted subcutaneously (SQ). The dose administered is the equivalent of the 2 mg/kg rat dose (=600 μg/day). The mouse equivalent based on body surface area is 4 mg/kg, i.e., about ˜100 μg/d for a 25 g mouse. No. 2001 mini-osmotic pumps (Durect) are prefilled with 200 μL murine Gal-3C (4 mg/ml) in PBS with 8 mg/ml lactose or vehicle only. Each pump delivers 1.0 μL/hour for 7 days. Pumps are removed after 7 days and new pumps are implanted as needed for dosing schedules described above. Alternatively, in method not requiring surgical implantation of a pump, Gal-3C (2-6 mg/ml) are administered once daily (0.5 ml) by intraperitoneal (IP) injection.

Physical and Biochemical Measurements.

Animals are weighed prior to injury, twice a week following liver injury (i.e., BDL or CCl₄ exposure), and again just prior to euthanasia. Plasma samples (100 μL) are obtained from tail vein blood weekly and by cardiac puncture postmortem. Plasma is analyzed for galectin-3, bilirubin, alanine aminotransferase (ALT), aspartate transaminase (AST), and alkaline phosphatase (ALP) activity (42,48) and ALP is assayed by colorimetric assay (Abcam, Cambridge, UK). Bilirubin levels are assessed with a commercially available kit (Sigma-Aldrich) according to manufacturer's instructions.

Histological Analyses.

Livers are removed from the mice after euthanasia, weighed, and liver samples are obtained for total RNA analysis. Portions of the livers are fixed in 4% PFA, paraffin-embedded and sectioned (4 μM) as described (42). Quantitative analysis of Picosirius stained sections using OPENLAB software to quantify fibrosis are performed. The liver sections are stained using 0.1% Sirius red and fast green in saturated picric acid as reported (6,32). Immunohistochemical (IHC) staining is performed with anti-α-SMA antibodies, (Sigma-Aldrich) and antibody binding is detected using Vectastain Kit (Vector Labs) as described (32)

Real-Time (RT) PCR.

Total RNA is isolated from liver pieces. Procollagen-1α (I), α-SMA, TNF-α, MCP-1, IL-1β, IL-6, 11-4, IL-10, Galectin-3, and β-Actin RNA levels are quantified by RT-PCR as reported (32).

### Example 4. Fusion of the Mouse Analog of Galectin-3C with Domain 3 from Human Serum Albumin

Rationale.

The pharmacokinetics of proteins and peptides is governed by the parameters of absorption via the lymphatic system (49), biodistribution, metabolism, and elimination which occurs through enzymatic cleavage by proteases and peptidases (50-52). The most common routes of clearance for proteins and peptides include endocytosis and membrane transport-mediated clearance by liver hepatocytes for larger proteins, and glomerular filtration by the kidney for smaller proteins and peptides (50,53). Many of the therapeutic proteins and peptides approved by the US Food and Drug Administration (FDA) for a wide variety of indications have less than optimal pharmacokinetic properties, often because they are smaller than the kidney filtration cutoff of around 70 kDa (53,54) and/or are subject to metabolic turnover by peptidases, which significantly limits their in vivo half-life (52). Furthermore, the charge of the protein or peptide plays an important role in the glomerular filtration; for instance, negatively charged small proteins or peptides may be eliminated less readily than neutral ones because of repulsion by the negatively charged basement membrane of the kidney (53,55). On the other hand, cationic peptides and small proteins tend to be eliminated even more quickly (55). In order to increase the half-life of the small proteins, two strategies have been generally employed—either increasing the size and the hydrodynamic radius of the therapeutic protein or increasing its negative charge. A third and the most commonly used strategy to extend the half-life of therapeutic proteins is protein fusion technology. Some natural proteins with long half-life have been used as fusion partners to enhance the circulating half-life of drugs, such as IgG-Fc, transferrin, and human serum albumin (HSA) (56-61). Our therapeutic protein Galectin-3C has a half-life of 3 hours and a molecular weight of ˜16 kDa. It is therefore smaller than the kidney cutoff of 70 kDa, which partly explains its relatively short half-life of about 3 h in rodents. Indeed, in some case, the half-life of proteins in human serum can be roughly correlated with their size (62) as shown below:

| Protein | Half-life (hours) | Molecular mass (kDa) |
|---|---|---|
| IGF-1 | 0.17 | 8 |
| IL-2 | 1.7 | 15 |
| G-CSF | 2 | 20 |
| IFN-alpha | 5 | 19 |
| HSA | 456 | 67 |

In view of the above, our objective is to carry out a genetic fusion of Gal-3C to human serum albumin (HSA) a naturally long-half-life serum protein or to one of its domains, i.e., domain I (1DHSA), domain II (2DHSA) or domain III (3DHSA). This albumin fusion could deflect human Gal-3C from rapid in vivo clearance without impairing its galectin-3 inhibition properties and widens its potential therapeutic window. As is shown in the table above, human serum albumin a long half-life of 456 hours or 19 days that has been determined to be due to its binding to the neonatal Fc receptor (FcRn). In general, FcRn can capture HSA in a pH-dependent manner, which protects HSA from normal lysosomal degradation after being taken into cells (63). Further investigation indicated this long circulation persistence of HSA is enabled by the interaction of the neonatal Fc receptor with domain III, which is necessary and sufficient for binding to FcRn. The histidine residues in 3DHSA apparently could dominate the interaction between HSA and FcRn (63-65). Thus, 3DHSA is a good candidate as a potential fusion partner for our therapeutic protein Gal-3C.

In order to prolong the half-life of Gal-3C in vivo we will genetically fuse it with HSA or preferably each one of its domains 1DHSA, 2DHSA or 3DHSA and then express the HSA-Gal-3C, 1DHSA-Gal3C, 2DHSA-Gal3C or 3DHSA-Gal3C fusion protein in E. coli. The biological activity and pharmacokinetic property of the purified fusion protein will also be studied in each case. Below is a summary of the reported physicochemical properties of the HSA and its recombinant domains (66).

| Protein | Molecular mass kDa | AA Residue Range | Isoelectric point, pI |
|---|---|---|---|
| HSA | 66.47 | 1-585 | 5.8 |
| 1DHSA | 22.96 | 1-197 | 5.2 |
| 2DHSA | 22.41 | 189-385 | 5.4 |
| 3DHSA | 23.32 | 381-585 | 6.6 |

Below we describe the strategy for producing the genetic fusion of Gal-3C to 3DHSA. However, this is also the general method for the genetic fusion HSA or its other two domains 1DHSA or 2DHSA. The fusion of Gal-3C with a domain of HSA is preferable as being smaller than HSA, there is less potential for hindrance with the binding of Gal-3C to carbohydrates.

A preliminary study of genetic fusion of mouse Gal-3C to 3DHSA was carried out in order to evaluate the expression of the fusion protein in 3 different E. coli strains and 2 two different incubation conditions, 37° C. for 4 hours and 15° C. for 16 hours. These conditions were used to generate the mouse analog of Gal-3C through its N-terminus to the C-terminus of 3DHSA, in one case, and mouse Gal-3C linked through its C-terminus to the N-terminus of 3DHSA. The target sequence was subcloned into E. coli expression vector pET30a using Nde I and Hind III. Three types of E. coli competent cells were transformed with the recombinant plasmid: E. coli Arctic Express (DE3), BL21(DE3) and Rosetta™ 2(DE3). A single colony was then inoculated into LB medium containing kanamycin; cultures were incubated in 37° C. at 200 rpm. Once cell density reached to OD=0.6-0.8 at 600 nm, 0.5 mM IPTG is introduced for induction. SDS-PAGE is used to monitor the expression.

3DHSA linked through its C-terminus to the N-terminus of mouse Gal-3C

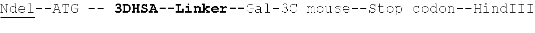

Structure of protein: Length = 355 amino acids, MW = 39960.9 Predicted pl = 8.17 vector:
pET30a
(SEQ ID NO: 10)
MLEKCCAAADPHECYAKVFDEFKPLVEEPQNLIKQNCELFEQLGEYKFQNALLVRYTKKVPQ
VSTPTLVEVSRNLGKVGSKCCKHPEAKRMPCAEDYLSVVLNQLCVLHEKTPVSDRVTKCCTE
SLVNRRPCFSALEVDETYVPKEFNAETFTFHADICTLSEKERQIKKQTALVELVKHKPKATK

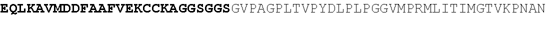

RIVLDFRRGNDVAFHFNPRFNENNRRVIVCNTKQDNNWGKEERQSAFPFESGKPFKIQVLVE

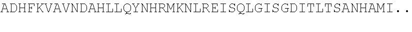

Structure of DNA: Length = 1080 bp
(SEQ ID NO: 11)
CATATGTTAGAAAAATGTTGTGCTGCAGCTGATCCCCACGAATGTTACGCAAAGGTGTTTGA
CGAGTTTAAGCCGCTGGTTGAAGAACCGCAGAACCTGATCAAACAAAACTGCGAGCTGTTCG
AACAGCTGGGCGAGTACAAGTTTCAAAACGCGCTGCTGGTGCGTTATACCAAGAAAGTGCCG
CAGGTTAGCACCCCGACCCTGGTGGAAGTTAGCCGTAACCTGGGTAAAGTTGGCAGCAAATG
CTGCAAACATCCGGAGGCGAAGCGTATGCCGTGCGCGGAAGACTACCTGAGCGTGGTTCTGA
ACCAACTGTGCGTGCTGCACGAGAAAACCCCGGTGAGCGATCGTGTTACCAAGTGCTGCACC
GAAAGCCTGGTTAACCGTCGTCCGTGCTTCAGCGCGCTGGAAGTGGACGAAACCTATGTTCC
GAAAGAGTTTAACGCGGAAACCTTCACCTTTCACGCGGATATCTGCACCCTGAGCGAGAAGG
AACGTCAGATTAAGAAACAAACCGCGCTGGTTGAGCTGGTTAAGCACAAACCGAAGGCGACC
AAAGAACAGCTGAAGGCGGTGATGGACGATTTCGCGGCGTTTGTTGAGAAATGCTGCAAGGC

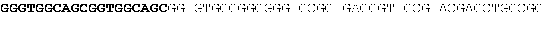

TGCCGGGTGGCGTGATGCCGCGTATGCTGATCACCATTATGGGTACCGTGAAACCGAACGCG
AACCGTATCGTTCTGGACTTCCGTCGTGGCAACGATGTTGCGTTCCACTTTAACCCGCGTTT
TAACGAGAACAACCGTCGTGTGATTGTTTGCAACACCAAACAGGATAACAACTGGGGTAAAG
AGGAGCGTCAAAGCGCGTTCCCGTTTGAGAGCGGCAAACCGTTCAAGATTCAGGTGCTGGTT
GAAGCGGACCACTTTAAAGTGGCGGTTAACGATGCGCACCTGCTGCAATATAACCACCGTAT
GAAGAACCTGCGTGAAATTAGCCAACTGGGCATTAGCGGTGACATTACCCTGACCAGCGCGA

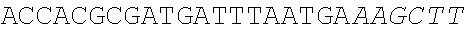

Mouse Gal-3C linked through its C-terminus of the N-terminus of 3DHSA

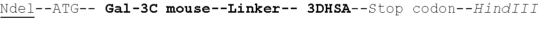

Structure of protein: Length = 355 MW = 39954.0 Predicted pl = 8.17 vector: pET30a
(SEQ ID NO: 12)
MGVPAGPLTVPYDLPLPGGVMPRMLITIMGTVKPNANRIVLDFRRGNDVAFHFNPRFNENNR
RVIVCNTKQDNNWGKEERQSAFPFESGKPFKIQVLVEADHFKVAVNDAHLLQYNHRMKNLRE

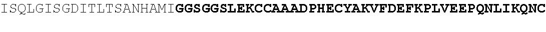

ELFEQLGEYKFQNALLVRYTKKVPQVSTPTLVEVSRNLGKVGSKCCKHPEAKRMPCAEDYLS
VVLNQLCVLHEKTPVSDRVTKCCTESLVNRRPCFSALEVDETYVPKEFNAETFTFHADICTL

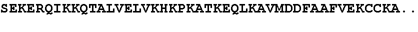

Structure of DNA: Length = 1080 bp
(SEQ ID NO: 13)
CATATGGGTGTGCCGGCGGGTCCGCTGACCGTTCCGTACGACCTGCCGCTGCCGGGTGGCGT
GATGCCGCGTATGCTGATCACCATTATGGGTACCGTGAAACCGAACGCGAACCGTATCGTTC
TGGACTTCCGTCGTGGCAACGATGTTGCGTTCCACTTTAACCCGCGTTTTAACGAGAACAAC
CGTCGTGTGATTGTTTGCAACACCAAACAGGATAACAACTGGGGTAAAGAGGAGCGTCAAAG
CGCGTTCCCGTTTGAGAGCGGCAAACCGTTCAAGATTCAGGTGCTGGTTGAAGCGGACCACT
TTAAAGTGGCGGTTAACGATGCGCACCTGCTGCAATATAACCACCGTATGAAGAACCTGCGT
GAAATTAGCCAACTGGGCATTAGCGGTGACATTACCCTGACCAGCGCGAACCACGCGATGAT

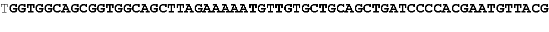

CAAAGGTGTTTGACGAGTTTAAGCCGCTGGTTGAAGAACCGCAGAACCTGATCAAACAAAAC
TGCGAGCTGTTCGAACAGCTGGGCGAGTACAAGTTTCAAAACGCGCTGCTGGTGCGTTATAC
CAAGAAAGTGCCGCAGGTTAGCACCCCGACCCTGGTGGAAGTTAGCCGTAACCTGGGTAAAG
TTGGCAGCAAATGCTGCAAACATCCGGAGGCGAAGCGTATGCCGTGCGCGGAAGACTACCTG
AGCGTGGTTCTGAACCAACTGTGCGTGCTGCACGAGAAAACCCCGGTGAGCGATCGTGTTAC
CAAGTGCTGCACCGAAAGCCTGGTTAACCGTCGTCCGTGCTTCAGCGCGCTGGAAGTGGACG
AAACCTATGTTCCGAAAGAGTTTAACGCGGAAACCTTCACCTTTCACGCGGATATCTGCACC
CTGAGCGAGAAGGAACGTCAGATTAAGAAACAAACCGCGCTGGTTGAGCTGGTTAAGCACAA
ACCGAAGGCGACCAAAGAACAGCTGAAGGCGGTGATGGACGATTTCGCGGCGTTTGTTGAGA

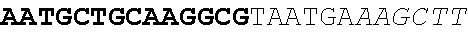

TABLE 3
Summary of results from analysis of expression
of Gal-3C fusions with 3DHSA

| | | | Expression |
|---|---|---|---|
| | E. coli | Best expression | level |
| | Strain | conditions | (mg/ml) |
| 3DHSA -Gal3C | Arctic | induction for 16 h | 30 |
| 3DHSA linked through | Express(DE3) | at 15° C. | |
| its C-terminus to the | BL21(DE3) | induction for 16 h | 35 |
| N-terminus of mouse | | at 15° C. | |
| Gal-3C | Rosetta ™ | induction for 16 h | 10 |
| | 2(DE3) | at 15° C. | |
| Gal3C- 3DHSA | Arctic | / | <1 |
| Gal-3C linked through | Express(DE3) | | |
| its C-terminus of the | BL21(DE3) | / | <1 |
| N-terminus to the | Rosetta ™ | / | <1 |
| 3DHSA | 2(DE3) | | |

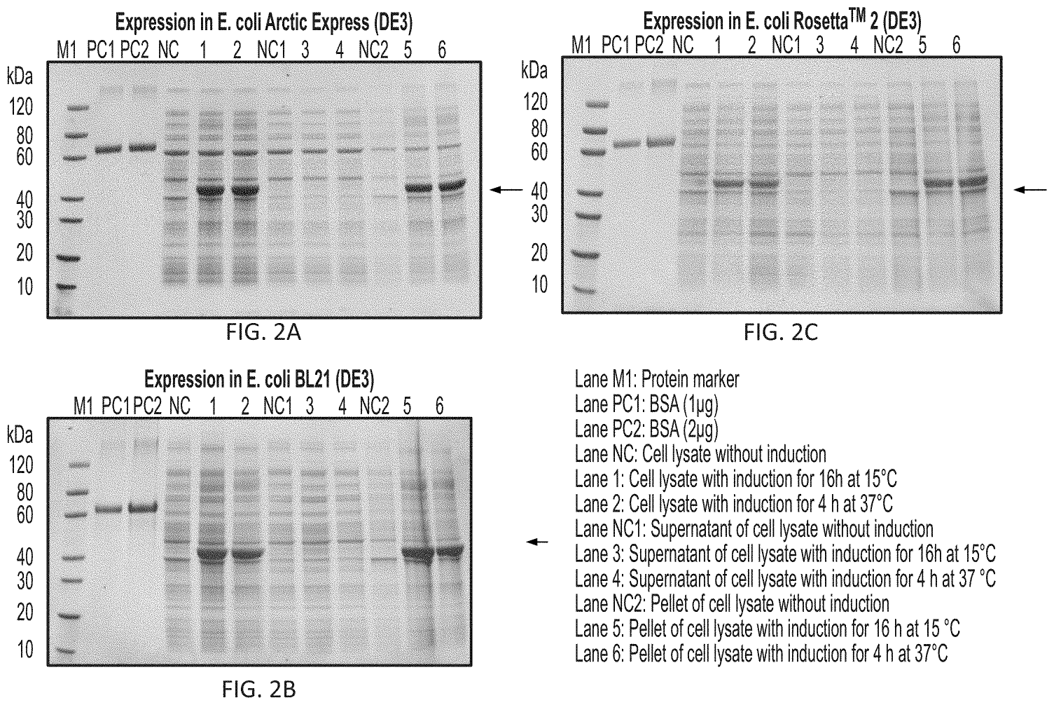

The linkage of 3DHSA through its C-terminus to N-terminus of mouse Gal-3C as shown in FIG. 2A-2C resulted in expression of the fusion protein in all the three strains of E. coli. However the expression level with the Rosetta™ 2(DE3) was the lowest and corresponded to three times less that the level of expression obtained with Arctic Express (DE3) or BL21 (DE3) strains. These two later strains produced about equivalent level of expression of the fusion protein at 30 and 35 mg/ml respectively.

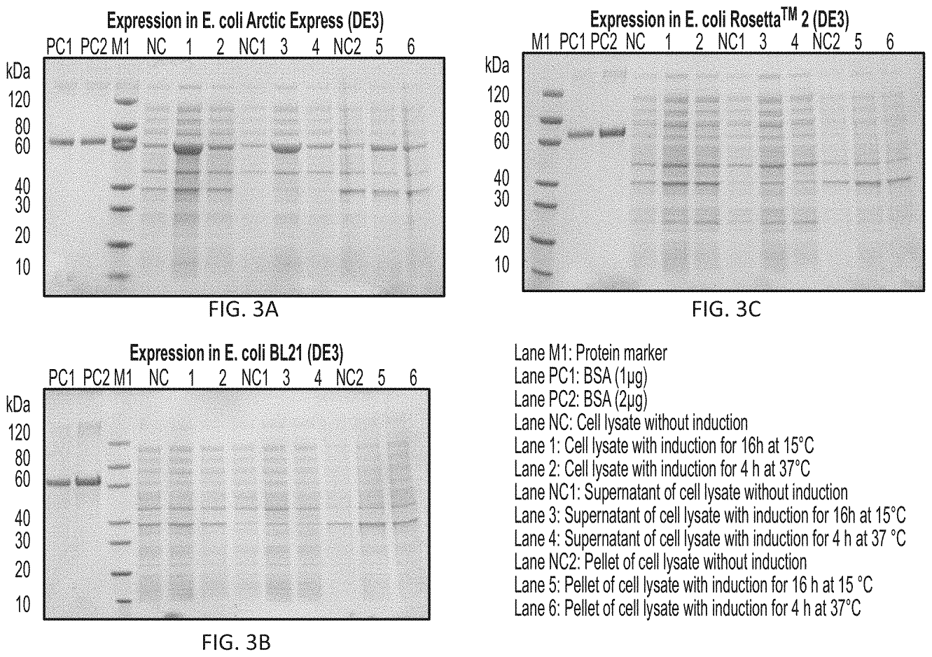

In the case of Gal-3C linked through its C-terminus of the N-terminus of 3DHSA very poor expression of the fusion protein as shown in FIG. 3A-3C was observed with all the three E. coli strains and with all the incubation conditions.

In view of the above results obtained with the fusion of mouse Gal-3C and 3DHSA, additional studies will be conducted to make the human Gal3C-3DHSA fusion protein with an extended half-life. Initial experiments will: (i) avoid linking human Gal-3C to 3DHSA using the C-terminus of Gal-3C; (ii) link the 3DHSA through its C-terminus to the N-terminus of Gal-3C (iii) use the E. coli strain BL21(DE3), and (iv) use the expression conditions of induction for 16 hours at 15° C.

To evaluate the half-life extension in vivo, we will determine the serum levels at 10-20 time points with the last being at least one week after intravenous or subcutaneous injection of mouse Gal-3C and the D3HSA derivatives of Gal-3C in humanized FcRn (Hu-FcRn) mice available from Jackson Laboratories (Bar Harbor, Me.). This mouse enables obtaining clinically-relevant PK data related to drug half-life because it expresses the Hu-FcRn.

### Example 5. Fusion of Human Galectin-3C with Domain 3 from Human Serum Albumin

In view of the above results obtained with the fusion of mouse Gal-3C and 3DHSA, we will generate a human Gal3C-3DHSA fusion protein. Human Gal-3C is a 16 kDa protein consisting of 143 amino acids. 3DHSA is a 23.32 kDa protein containing 204 amino acids. The molecular weight of the fused protein 3DHSA-Gal3C is ˜40 kDa. Three constructs will be generated:

1) Link 3DHSA through its C-terminus to the N-terminus of Gal-3C

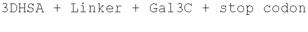

In this case a similar strategy as the one described above with mouse Gal-3C is used.
(SEQ ID NO: 14)
MLEKCCAAADPHECYAKVFDEFKPLVEEPQNLIKQNCELFEQLGEYKFQNALLVRYTKINPQV
STPTLVEVSRNLGKVGSKCCKHPEAKRMPCAEDYLSVVLNQLCVLHEKTPVSDRVTKCCTESL
VNRRPCFSALEVDETYVPKEFNAETFTFHADICTLSEKERQIKKQTALVELVKHKPKATKEQL

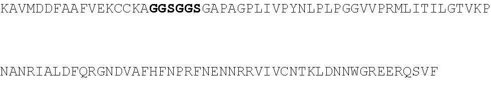

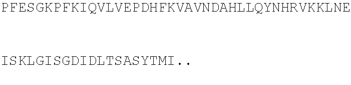

2) Link 3DHSA through its N-terminus to the N-terminus of Gal-3C

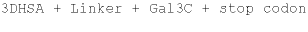

(SEQ ID NO: 15)
MAKCCKEVFAAFDDMVAKLQEKTAKPKHKVLEVLATQKKIQREKESLTCIDAHFTFTEANFE
KPVYTEDVELASFCPRRNVLSETCCKTVRDSVPTKEHLVCLQNLVVSLYDEACPMRKAEPHKC
CKSGVKGLNRSVEVLTPTSVQPVKKTYRVLLANQFKYEGLQEFLECNQKILNQPEEVLPKFEDF

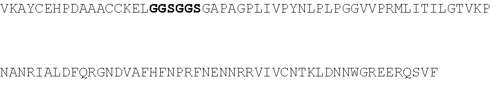

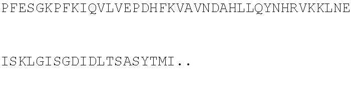

3) Link Gal-3C through its C-terminus to the C-terminus of 3DHSA

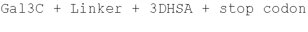

(SEQ ID NO: 16)
MGAPAGPLIVPYNLPLPGGVVPRMLITILGTVKPNANRIALDFQRGNDVAFHFN
PRFNENNRRVIVCNTKLDNNWGREERQSVFPFESGKPFKIQVLVEPDHFKVAVNDAH

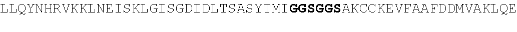

KTAKPKHKVLEVLATQKKIQREKESLTCIDAHFTFTEANFEKPVYTEDVELASFCPRRNVLSET
CCKTVRDSVPTKEHLVCLQNLVVSLYDEACPMRKAEPHKCCKSGVKGLNRSVEVLTPTSVQPV

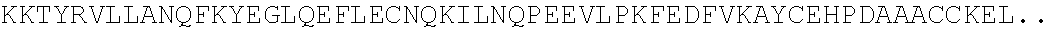

Cloning Strategy:

Subclone target sequence into E. coli expression vector pET30a using Nde I and Hind III. Briefly E. coli BL21(DE3) competent cells is transformed with the recombinant plasmid. A single colony is inoculated into LB medium containing kanamycin; cultures is incubated at 37° C. and 200 rpm. Once cell density reaches OD=0.6-0.8 at 600 nm, 0.5 mM IPTG is introduced for induction. SDS-PAGE is used to monitor the expression.

We have previously successfully used E. coli to produce human, porcine and mouse Gal-3C with very good yield. This was achieved without having a refolding step by growing the bacteria, centrifuging to obtain the bacterial pellet, lysing the pellet and purifying the Gal-3C present in the lysate by a purification step through an affinity column consisting of lactosyl-sepharose beads. The Gal-3C present in the lysate binds specifically to the lactosyl-sepharose beads and after several washes consisting of 4 to 5 column volumes with PBS the bound Gal-3C is eluted with an elution buffer containing lactose. Because the recombinant Gal-3C binds by itself to the lactosyl-sepharose column, it does not need to be tagged (by 6-His for example). In addition, there is no need for a refolding step since the Gal-3C produced is fully functional. We employ a similar procedure when we are producing the 3DHSA-Gal3C fusion protein using the same lactosyl-sepharose affinity column for purification, i.e., no need to tag the fused protein, and no need for a refolding step. We will evaluate the optimization of the following conditions with SDS-PAGE and Western blotting analysis:

| | | | |
|---|---|---|---|
| Condition 1: | LB medium | 15° C. for 16 h | BL21(DE 3) |
| Condition 2: | LB medium | 37° C. for 4 h | BL21(DE 3) |
| Condition 3: | LB medium | 15° C. for 16 h | BL21 Star ™ (DE3) |
| Condition 4: | LB medium | 37° C. for 4 h | BL21 Star ™ (DE3) |
| Condition 7: | TB medium | 15° C. for 16 h | BL21(DE 3) |
| Condition 8: | TB medium | 37° C. for 4 h | BL21(DE 3) |
| Condition 9: | TB medium | 15° C. for 16 h | BL21 Star ™ (DE3) |
| Condition 10: | TB medium | 37° C. for 4 h | BL21 Star ™ (DE3) |
| Condition 13: | Auto-induction | 15° C. for 16 h | BL21(DE 3) |
| | medium | | |
| Condition 14: | Auto-induction | 37° C. for 4 h | BL21(DE 3) |
| | medium | | |
| Condition 15: | Auto-induction | 15° C. for 16 h | BL21 Star ™ (DE3) |
| | medium | | |
| Condition 16: | Auto-induction | 37° C. for 4 h | BL21 Star ™ (DE3) |
| | medium | | |

However, high-level expression of the recombinant 3DHSA-Gal3C in E. coli may result in aggregation of the expressed protein molecules in the inclusion bodies and will pose a major challenge for large scale recovery of bioactive Gal-3C. Inclusion bodies need extensive processing involving isolation from cell, solubilization, refolding and purification to produce the bioactive proteins. In case we face this type of problem, we will explore the production of “nonclassical inclusion bodies” as described in the literature (67-70). This new approach uses low temperature enabling the formation of nonclassical inclusion bodies from which correctly folded protein can be readily extracted by nondenaturing solvents, such as mild detergents or low concentrations of polar solvents such DMSO and nondetergent sulfobetaines (69). An example of non-denaturing solubilization methods for non-classical inclusion bodies with no need for refolding uses Tris-HCl buffer, low concentration of DMSO, n-propanol and sarcosyl (68,71,72).

The Following Protocol is Used to Produce the Nonclassical Inclusion Bodies (69):
Express target protein under: 25° C. for 18 h (at least) to 24 h. The optimal conditions for the formation of the Non-Classical Inclusion Bodies is between 20 and 25° C. for 18 to 24 hours. The nonclassical inclusion bodies need to be formed slowly.
The inoculum will be grown overnight at 25° C. under shaking then transferred into the production media.
We will not use any temperature under 20° C. or above 25° C. because the nonclassical Inclusion Bodies will not form.
The cell pellet is lysed by sonication and inclusion bodies collected.
The inclusion bodies are washed twice, using 50 mM Tris/HCl, pH 8.0 buffer and suspended in lysis buffer (40 mM Tris/HCl, pH 8.0 buffer containing 0.2% N-lauroyl-sarcosine) solubilized at room temperature overnight with slow agitation
The supernatant of inclusion bodies is collected and analyzed by SDS-PAGE.
The fusion protein is purified using the lactosyl-sepharose affinity column as mentioned above.

To evaluate the half-life extension conferred by expression of HSA or one of its domains on Gal-3C in vivo, we will determine the serum levels at 10-20 time points with the last being at least one week after intravenous or subcutaneous injection of human Gal-3C and the D3HSA derivatives of human Gal-3C in humanized FcRn (Hu-FcRn) mice available from Jackson Laboratories (Bar Harbor, Me.). This mouse enables obtaining clinically-relevant PK data related to drug half-life because it expresses the Hu-FcRn.

### REFERENCES

1. Torok, N. J. 2008. Recent advances in the pathogenesis and diagnosis of liver fibrosis. J Gastroenterol. 43:315-321.
2. Gieling, R. G., Burt, A. D., and Mann, D. A. 2008. Fibrosis and cirrhosis reversibility—molecular mechanisms. Clin Liver Dis. 12:915-937, xi.
3. Kisseleva, T., and Brenner, D. A. 2006. Hepatic stellate cells and the reversal of fibrosis. J Gastroenterol Hepatol. 21 Suppl 3:S84-87.
4. Gutierrez-Ruiz, M. C., and Gomez-Quiroz, L. E. 2007. Liver fibrosis: searching for cell model answers. Liver Int. 27:434-439.
5. Atzori, L., Poli, G., and Perra, A. 2009. Hepatic stellate cell: a star cell in the liver. Int J Biochem Cell Biol. 41:1639-1642.
6. Henderson, N. C., Mackinnon, A. C., Farnworth, S. L., Poirier, F., Russo, F. P., Iredale, J. P., Haslett, C., Simpson, K. J., and Sethi, T. 2006. Galectin-3 regulates myofibroblast activation and hepatic fibrosis. Proc Natl Acad Sci USA. 103:5060-5065.
7. Jiang, J. X., Chen, X., Hsu, D., Baghy, K., Serizawa, N., Scott, F., Takada, Y., Takada, Y., Fukada, H., Chen, J., Devaraj, S., Adamson, R., Liu, F.-T., and Torok, N. J. 2012. Galectin-3 modulates phagocytosis-induced stellate cell activation and liver fibrosis in vivo. Amer. J. Physiol. 302:G439-446.
8. Wanninger, J., Weigert, J., Wiest, R., Bauer, S., Karrasch, T., Farkas, S., Scherer, M. N., Walter, R., Weiss, T. S., Hellerbrand, C., Neumeier, M., Schaffler, A., and Buechler, C. 2011. Systemic and hepatic vein galectin-3 are increased in patients with alcoholic liver cirrhosis and negatively correlate with liver function. Cytokine. 55:435-440.
9. Cunningham, B. C., and Wells, J. A. 1989. High-resolution epitope mapping of hGH-receptor interactions by alanine-scanning mutagenesis. Science. 244:1081-1085.
10. Smith, G. P. 1991. Surface presentation of protein epitopes using bacteriophage expression systems. Curr Opin Biotechnol. 2:668-673.
11. Balan, V., Nangia-Makker, P., Kho, D. H., Wang, Y., and Raz, A. 2012. Tyrosine-phosphorylated galectin-3 protein is resistant to prostate-specific antigen (PSA) cleavage. J Biol Chem. 287:5192-5198.
12. Lepur, A., Carlsson, M., Novak, R., Dumic, J., Nilsson, U., and Leffler, H. 2012. Galectin-3 endocytosis by carbohydrate independent and dependent pathways in different macrophage like cell types. Biochim Biophys Acta. 1820:804-818.
13. Miyakawa, N., Nishikawa, M., Takahashi, Y., Ando, M., Misaka, M., Watanabe, Y., and Takakura, Y. 2013. Gene delivery of albumin binding peptide-interferon-gamma fusion protein with improved pharmacokinetic properties and sustained biological activity. J Pharm Sci. 102:3110-3118.
14. Dennis, M. S., Zhang, M., Meng, Y. G., Kadkhodayan, M., Kirchhofer, D., Combs, D., and Damico, L. A. 2002. Albumin binding as a general strategy for improving the pharmacokinetics of proteins. J Biol Chem. 277:35035-35043.
15. Milla, P., Dosio, F., and Cattel, L. 2012. PEGylation of proteins and liposomes: a powerful and flexible strategy to improve the drug delivery. Curr Drug Metab. 13:105-119.
16. Constantinou, A., Epenetos, A. A., Hreczuk-Hirst, D., Jain, S., and Deonarain, M. P. 2008. Modulation of antibody pharmacokinetics by chemical polysialylation. Bioconjug Chem. 19:643-650.
17. Gregoriadis, G., Fernandes, A., Mital, M., and McCormack, B. 2000. Polysialic acids: potential in improving the stability and pharmacokinetics of proteins and other therapeutics. Cell Mol Life Sci. 57:1964-1969.
18. Schlapschy, M., Binder, U., Borger, C., Theobald, I., Wachinger, K., Kisling, S., Haller, D., and Skerra, A. 2013. PASylation: a biological alternative to PEGylation for extending the plasma half-life of pharmaceutically active proteins. Protein Eng Des Sel. 26:489-501.
19. Schellenberger, V., Wang, C. W., Geething, N. C., Spink, B. J., Campbell, A., To, W., Scholle, M. D., Yin, Y., Yao, Y., Bogin, O., Cleland, J. L., Silverman, J., and Stemmer, W. P. 2009. A recombinant polypeptide extends the in vivo half-life of peptides and proteins in a tunable manner. Nat Biotechnol. 27:1186-1190.
20. Trussel, S., Dumelin, C., Frey, K., Villa, A., Buller, F., and Neri, D. 2009. New strategy for the extension of the serum half-life of antibody fragments. Bioconjug Chem. 20:2286-2292.
21. Osol, A. 1980. Remington's Pharmaceutical Sciences, 16th ed., Mack Pub
22. Ju, C., and Tacke, F. 2016. Hepatic macrophages in homeostasis and liver diseases: from pathogenesis to novel therapeutic strategies. Cell Mol Immunol. 13:316-327.
23. John, C. M., Gaur, M., Springer, M. L., and Wang, X. 2017. Methods and compositions for preventing and treating damage to the heart. MandalMed, Inc., The Regents of the University of California, USA. 10369195
24. Das, A., Shergill, U., Thakur, L., Sinha, S., Urrutia, R., Mukhopadhyay, D., and Shah, V. H. 2010. Ephrin B2/EphB4 pathway in hepatic stellate cells stimulates Erk-dependent VEGF production and sinusoidal endothelial cell recruitment. Am J Physiol Gastrointest Liver Physiol. 298:G908-915.
25. Jiang, J. X., Mikami, K., Venugopal, S., Li, Y., and Torok, N. J. 2009. Apoptotic body engulfment by hepatic stellate cells promotes their survival by the JAK/STAT and Akt/NF-kappaB-dependent pathways. J Hepatol. 51:139-148.
26. Feizi, T., Solomon, J. C., Yuen, C. T., Jeng, K. C., Frigeri, L. G., Hsu, D. K., and Liu, F. T. 1994. The adhesive specificity of the soluble human lectin, IgE-binding protein, toward lipid-linked oligosaccharides. Presence of the blood group A, B, B-like, and H monosaccharides confers a binding activity to tetrasaccharide (lacto-N-tetraose and lacto-N-neotetraose) backbones. Biochemistry. 33:6342-6349.
27. Mirandola, L., Yu, Y., Chui, K., Jenkins, M. R., Cobos, E., John, C. M., and Chiriva-Internati, M. 2011. Galectin-3C Inhibits Tumor Growth and Increases the Anticancer Activity of Bortezomib in a Murine Model of Human Multiple Myeloma. PLoS One. 6:e21811; PMC3135605.
28. Atorrasagasti, C., Aquino, J. B., Hofman, L., Alaniz, L., Malvicini, M., Garcia, M., Benedetti, L., Friedman, S. L., Podhajcer, O., and Mazzolini, G. 2011. SPARC downregulation attenuates the profibrogenic response of hepatic stellate cells induced by TGF-beta1 and PDGF. Am J Physiol Gastrointest Liver Physiol. 300:G739-748.
29. Coffelt, S. B., Waterman, R. S., Florez, L., Honer zu Bentrup, K., Zwezdaryk, K. J., Tomchuck, S. L., LaMarca, H. L., Danka, E. S., Morris, C. A., and Scandurro, A. B. 2008. Ovarian cancers overexpress the antimicrobial protein hCAP-18 and its derivative LL-37 increases ovarian cancer cell proliferation and invasion. Int J Cancer. 122:1030-1039.
30. Vogel, D. Y., Heijnen, P. D., Breur, M., de Vries, H. E., Tool, A. T., Amor, S., and Dijkstra, C. D. 2014. Macrophages migrate in an activation-dependent manner to chemokines involved in neuroinflammation. J Neuroinflammation. 11:23.
31. Lagana, A., Goetz, J. G., Cheung, P., Raz, A., Dennis, J. W., and Nabi, I. R. 2006. Galectin binding to Mgat5-modified N-glycans regulates fibronectin matrix remodeling in tumor cells. Mol Cell Biol. 26:3181-3193.
32. Cui, W., Matsuno, K., Iwata, K., Ibi, M., Matsumoto, M., Zhang, J., Zhu, K., Katsuyama, M., Torok, N. J., and Yabe-Nishimura, C. 2011. NOX1/nicotinamide adenine dinucleotide phosphate, reduced form (NADPH) oxidase promotes proliferation of stellate cells and aggravates liver fibrosis induced by bile duct ligation. Hepatology. 54:949-958.
33. Saha, B., Kodys, K., and Szabo, G. 2016. Hepatitis C Virus-Induced Monocyte Differentiation Into Polarized M2 Macrophages Promotes Stellate Cell Activation via TGF-beta. Cell Mol Gastroenterol Hepatol. 2:302-316 e308.
34. Saraswati, S., Block, A. S., Davidson, M. K., Rank, R. G., Mahadevan, M., and Diekman, A. B. 2010. Galectin-3 is a substrate for prostate specific antigen (PSA) in human seminal plasma. Prostate. 71:197-208.
35. Ferrante, C. J., and Leibovich, S. J. 2012. Regulation of Macrophage Polarization and Wound Healing. Adv Wound Care (New Rochelle). 1:10-16.
36. Novak, R., Dabelic, S., and Dumic, J. 2012. Galectin-1 and galectin-3 expression profiles in classically and alternatively activated human macrophages. Biochim Biophys Acta. 1820:1383-1390.
37. MacKinnon, A. C., Farnworth, S. L., Hodkinson, P. S., Henderson, N. C., Atkinson, K. M., Leffler, H., Nilsson, U. J., Haslett, C., Forbes, S. J., and Sethi, T. 2008. Regulation of alternative macrophage activation by galectin-3. J Immunol. 180:2650-2658.
38. Liu, S. B., Ikenaga, N., Peng, Z. W., Sverdlov, D. Y., Greenstein, A., Smith, V., Schuppan, D., and Popov, Y. 2016. Lysyl oxidase activity contributes to collagen stabilization during liver fibrosis progression and limits spontaneous fibrosis reversal in mice. FASEB J. 30:1599-1609.
39. Vartak, N., Damle-Vartak, A., Richter, B., Dirsch, O., Dahmen, U., Hammad, S., and Hengstler, J. G. 2016. Cholestasis-induced adaptive remodeling of interlobular bile ducts. Hepatology. 63:951-964.
40. Abshagen, K., Konig, M., Hoppe, A., Muller, I., Ebert, M., Weng, H., Holzhutter, H. G., Zanger, U. M., Bode, J., Vollmar, B., Thomas, M., and Dooley, S. 2015. Pathobiochemical signatures of cholestatic liver disease in bile duct ligated mice. BMC Syst Biol. 9:83.
41. Iredale, J. P. 2007. Models of liver fibrosis: exploring the dynamic nature of inflammation and repair in a solid organ. J Clin Invest. 117:539-548.
42. Jiang, J. X., Venugopal, S., Serizawa, N., Chen, X., Scott, F., Li, Y., Adamson, R., Devaraj, S., Shah, V., Gershwin, M. E., Friedman, S. L., and Torok, N. J. 2010. Reduced nicotinamide adenine dinucleotide phosphate oxidase 2 plays a key role in stellate cell activation and liver fibrogenesis in vivo. Gastroenterology. 139:1375-1384.
43. Liedtke, C., Luedde, T., Sauerbruch, T., Scholten, D., Streetz, K., Tacke, F., Tolba, R., Trautwein, C., Trebicka, J., and Weiskirchen, R. 2013. Experimental liver fibrosis research: update on animal models, legal issues and translational aspects. Fibrogenesis Tissue Repair. 6:19.
44. Tsai, J. H., Liu, J. Y., Wu, T. T., Ho, P. C., Huang, C. Y., Shyu, J. C., Hsieh, Y. S., Tsai, C. C., and Liu, Y. C. 2008. Effects of silymarin on the resolution of liver fibrosis induced by carbon tetrachloride in rats. J Viral Hepat. 15:508-514.
45. Muriel, P., Moreno, M. G., Hernandez Mdel, C., Chavez, E., and Alcantar, L. K. 2005. Resolution of liver fibrosis in chronic CCl4 administration in the rat after discontinuation of treatment: effect of silymarin, silibinin, colchicine and trimethylcolchicinic acid. Basic Clin Pharmacol Toxicol. 96:375-380.
46. John, C. M., Leffler, H., Kahl-Knutsson, B., Svensson, I., and Jarvis, G. A. 2003. Truncated galectin-3 inhibits tumor growth and metastasis in orthotopic nude mouse model of human breast cancer. Clin Cancer Res. 9:2374-2383.
47. Zhan, S. S., Jiang, J. X., Wu, J., Halsted, C., Friedman, S. L., Zern, M. A., and Torok, N. J. 2006. Phagocytosis of apoptotic bodies by hepatic stellate cells induces NADPH oxidase and is associated with liver fibrosis in vivo. Hepatology. 43:435-443.
48. Abu-Elsaad, N. M., and Elkashef, W. F. 2016. Modified citrus pectin stops progression of liver fibrosis by inhibiting galectin-3 and inducing apoptosis of stellate cells. Can J Physiol Pharmacol. 94:554-562.
49. Supersaxo, A., Hein, W. R., and Steffen, H. 1990. Effect of molecular weight on the lymphatic absorption of water-soluble compounds following subcutaneous administration. Pharm Res. 7:167-169.
50. Diao, L., and Meibohm, B. 2013. Pharmacokinetics and pharmacokinetic-pharmacodynamic correlations of therapeutic peptides. Clin Pharmacokinet. 52:855-868.
51. Holst, J. J. 2007. The physiology of glucagon-like peptide 1. Physiol Rev. 87:1409-1439.
52. Werle, M., and Bernkop-Schnurch, A. 2006. Strategies to improve plasma half life time of peptide and protein drugs. Amino Acids. 30:351-367.
53. Caliceti, P., and Veronese, F. M. 2003. Pharmacokinetic and biodistribution properties of poly(ethylene glycol)-protein conjugates. Adv Drug Deliv Rev. 55:1261-1277.
54. Jevsevar, S., Kunstelj, M., and Porekar, V. G. 2010. PEGylation of therapeutic proteins. Biotechnol J. 5:113-128.
55. Tang, L., Persky, A. M., Hochhaus, G., and Meibohm, B. 2004. Pharmacokinetic aspects of biotechnology products. J Pharm Sci. 93:2184-2204.
56. Strohl, W. R. 2015. Fusion Proteins for Half-Life Extension of Biologics as a Strategy to Make Biobetters. BioDrugs. 29:215-239.
57. Halpern, W., Riccobene, T. A., Agostini, H., Baker, K., Stolow, D., Gu, M. L., Hirsch, J., Mahoney, A., Carrell, J., Boyd, E., and Grzegorzewski, K. J. 2002. Albugranin, a recombinant human granulocyte colony stimulating factor (G-CSF) genetically fused to recombinant human albumin induces prolonged myelopoietic effects in mice and monkeys. Pharm Res. 19:1720-1729.
58. Bai, Y., Ann, D. K., and Shen, W. C. 2005. Recombinant granulocyte colony-stimulating factor-transferrin fusion protein as an oral myelopoietic agent. Proc Natl Acad Sci USA. 102:7292-7296.
59. Cox, G. N., Smith, D. J., Carlson, S. J., Bendele, A. M., Chlipala, E. A., and Doherty, D. H. 2004. Enhanced circulating half-life and hematopoietic properties of a human granulocyte colony-stimulating factor/immunoglobulin fusion protein. Exp Hematol. 32:441-449.
60. O'Connor-Semmes, R. L., Lin, J., Hodge, R. J., Andrews, S., Chism, J., Choudhury, A., and Nunez, D. J. 2014. GSK2374697, a novel albumin-binding domain antibody (AlbudAb), extends systemic exposure of exendin-4: first study in humans—PK/PD and safety. Clin Pharmacol Ther. 96:704-712.
61. Andersen, J. T., Pehrson, R., Tolmachev, V., Daba, M. B., Abrahmsen, L., and Ekblad, C. 2011. Extending half-life by indirect targeting of the neonatal Fc receptor (FcRn) using a minimal albumin binding domain. J Biol Chem. 286:5234-5241.
62. Chaudhury, C., Brooks, C. L., Carter, D. C., Robinson, J. M., and Anderson, C. L. 2006. Albumin binding to FcRn: distinct from the FcRn-IgG interaction. Biochemistry. 45:4983-4990.
63. Roopenian, D. C., and Akilesh, S. 2007. FcRn: the neonatal Fc receptor comes of age. Nat Rev Immunol. 7:715-725.
64. Andersen, J. T., Daba, M. B., and Sandlie, I. 2010. FcRn binding properties of an abnormal truncated analbuminemic albumin variant. Clin Biochem. 43:367-372.
65. Baker, K., Qiao, S. W., Kuo, T., Kobayashi, K., Yoshida, M., Lencer, W. I., and Blumberg, R. S. 2009. Immune and non-immune functions of the (not so) neonatal Fc receptor, FcRn. Semin Immunopathol. 31:223-236.
66. Dockal, M., Carter, D. C., and Ruker, F. 1999. The three recombinant domains of human serum albumin. Structural characterization and ligand binding properties. J Biol Chem. 274:29303-29310.
67. Slouka, C., Kopp, J., Spadiut, O., and Herwig, C. 2019. Perspectives of inclusion bodies for bio-based products: curse or blessing? Appl Microbiol Biotechnol. 103:1143-1153.
68. Singh, A., Upadhyay, V., Upadhyay, A. K., Singh, S. M., and Panda, A. K. 2015. Protein recovery from inclusion bodies of Escherichia coli using mild solubilization process. Microb Cell Fact. 14:41.
69. Jevsevar, S., Gaberc-Porekar, V., Fonda, I., Podobnik, B., Grdadolnik, J., and Menart, V. 2005. Production of nonclassical inclusion bodies from which correctly folded protein can be extracted. Biotechnol Prog. 21:632-639.
70. Peternel, Š. (2010) Designing non-classical inclusion bodies. in Ribosomal Proteins and Protein Engineering Design, Selection and Applications (Ortendhal, V., and Sal, H. eds.), Nova Science Publishers, Hauppauge, N.Y. pp 1-20
71. Gatti-Lafranconi, P., Natalello, A., Ami, D., Doglia, S. M., and Lotti, M. 2011. Concepts and tools to exploit the potential of bacterial inclusion bodies in protein science and biotechnology. FEBS J. 278:2408-2418.
72. Lu, S. C., and Lin, S. C. 2012. Recovery of active N-acetyl-D-glucosamine 2-epimerase from inclusion bodies by solubilization with non-denaturing buffers. Enzyme Microb Technol. 50:65-70.

1. A method of reducing liver fibrosis in a subject in need thereof comprising administering a therapeutically effective amount of Galectin-3C (Gal-3C) to the subject.

2. A method of treating liver fibrosis in a subject in need thereof comprising administering a therapeutically effective amount of Galectin-3C (Gal-3C) to the subject.

3. A method of reversing liver fibrosis in a subject in need thereof comprising administering a therapeutically effective amount of Galectin-3C (Gal-3C) to the subject.

4. The method of any one of claims 1-3, wherein the Gal-3C is produced by culturing a host cell comprising a nucleic acid encoding the Gal-3C under conditions where the Gal-3C is expressed, and implanting the cells to administer the Gal-3C or harvesting the Gal-3C expressed by the host cell for administration.

5. The method of any one of claims 1-3, wherein the Gal-3C is fused to human serum albumin, or a domain thereof, to form a fusion protein.

6. The method of claim 5, wherein the fusion protein comprises a domain of human serum albumin selected from the group consisting of domain I (1DHSA), domain II (2DHSA) or domain III (3DHSA).

7. The method of claim 5, wherein the fusion protein comprises domain III (3DHSA) of human serum albumin.

8. The method of claim 7, wherein the C-terminus of 3DHSA is fused to the N-terminus of Gal-3C.

9. The method of claim 7, wherein the N-terminus of 3DHSA is fused to the N-terminus of Gal-3C.

10. The method of claim 7, wherein the C-terminus of 3DHSA is fused to the C-terminus of Gal-3C.

11. The method of any one of claims 5-10, wherein the fusion protein has an increased serum half-life compared to Gal-3C.

12. A fusion protein comprising Gal-3C fused to human serum albumin, or a domain thereof, wherein the fusion protein has an increased serum half-life compared to Gal-3C.

13. The fusion protein of claim 12, wherein the fusion protein comprises a domain of human serum albumin selected from the group consisting of domain I (1DHSA), domain II (2DHSA) or domain III (3DHSA).

14. The fusion protein of claim 13, wherein the C-terminus of 3DHSA is fused to the N-terminus of Gal-3C.

15. The fusion protein of claim 13, wherein the N-terminus of 3DHSA is fused to the N-terminus of Gal-3C.

16. The fusion protein of claim 13, wherein the C-terminus of 3DHSA is fused to the C-terminus of Gal-3C.
